(19) United States
(12) Patent Application Publication
(10) Pub. No.: US 2010/0154078 A1
(43) Pub. Date: Jun. 17, 2010

(54) TRANSCRIPTION FACTORS THAT ENHANCE TRAITS IN PLANT ORGANS

(75) Inventors: ANN L. T. POWELL, DAVIS, CA (US); OLIVER J. RATCLIFFE, OAKLAND, CA (US); T. LYNNE REUBER, SAN MATEO, CA (US); ALAN B. BENNETT, DAVIS, CA (US)

Correspondence Address: Mendel Biotechnology, Inc., 3935 Point Eden Way, Hayward, CA, 94545, US

(73) Assignee: MENDEL BIOTECHNOLOGY, INC., HAYWARD, CA (US)

(21) Appl. No.: 12/689,010

(22) Filed: Jan. 18, 2010

Related U.S. Application Data
(63) Continuation-in-part of application No. 11/986,992, filed on Nov. 26, 2007.
Application No. 11/986,992 is a division of application No. 10/412,699, filed on Apr. 10, 2003, now Pat. No. 7,345,217.
Application No. 10/412,699 is a continuation-in-part of application No. 10/302,267, filed on Nov. 22, 2002, now Pat. No. 7,223,904.
Application No. 10/302,267 is a division of application No. 09/506,720, filed on Feb. 17, 2000, now abandoned.
Application No. 10/412,699 is a continuation-in-part of application No. 09/713,994, filed on Nov. 16, 2000, now abandoned.
Application No. 09/713,994 is a continuation-in-part of application No. 11/479,226, filed on Jun. 30, 2006.
(60) Provisional application No. 61/146,204, filed on Jan. 21, 2009.
(60) Provisional application No. 60/129,450, filed on Apr. 15, 1999.

(51) Int. Cl. A01H 5/00; C12N 15/82

(52) U.S. Cl. 800/282; 800/298; 800/317.4; 800/284

(57) ABSTRACT

Expression of two Arabidopsis thaliana GARP-family transcription factors, AtGLK1, SEQ ID NO: 2, and AtGLK2, SEQ ID NO: 4, in tomato plants resulted in intensely green fruit that ripen to a normal red color. These Golden2-like (GLK) transcription factors were expressed under the control of several promoters in transgenic tomato lines. When AtGLK1 or AtGLK2 expression was regulated with the constitutive 35S promoter or with three promoters that enhanced expression in fruit tissues, the chlorophyll content of mature green fruit was increased by as much as 100%. The chloroplasts in green fruit expressing AtGLK1 or AtGLK2 developed earlier, were enlarged and had more extensive thylakoid granal development. In addition, expression of AtGLK1 or AtGLK2 resulted in increased starch accumulation in green fruit and higher levels of sugars in ripe fruit. In contrast to wild-type fruit, fruit expressing AtGLK1 developed full green color when they developed in the absence of light. Manipulation of the expression of GLK-like transcription factors in plants may provide a means for improving plant organ nutritional properties, particularly in plants or plant organs grown or maintained under low irradiance.

### RELATIONSHIP TO COPENDING APPLICATIONS

This application (the “present application”) claims the benefit of U.S. provisional application 61/146,204, filed Jan. 21, 2009 (pending). The present application is also a continuation-in-part of U.S. non-provisional application Ser. No. 11/986,992, filed Nov. 26, 2007 (pending), which is a division of U.S. non-provisional application Ser. No. 10/412,699, filed Apr. 10, 2003 (issued as U.S. Pat. No. 7,345,217), which is a continuation-in-part of U.S. non-provisional application Ser. No. 10/302,267, filed Nov. 22, 2002 (issued as U.S. Pat. No. 7,223,904), which is a division of U.S. non-provisional application Ser. No. 09/506,720, filed Feb. 17, 2000 (abandoned), which claims the benefit of U.S. provisional application 60/129,450, filed Apr. 15, 1999 (expired). U.S. non-provisional application Ser. No. 10/412,699 is also a continuation-in-part of U.S. non-provisional application Ser. No. 09/713,994, filed Nov. 16, 2000 (abandoned). The present application is also a continuation-in-part of U.S. non-provisional application Ser. No. 11/479,226, filed Jun. 30, 2006 (pending). The entire contents of each of these applications are hereby incorporated by reference.

### JOINT RESEARCH AGREEMENT

The claimed invention, in the field of functional genomics and the characterization of plant genes for the improvement of plants, was made by or on behalf of Mendel Biotechnology, Inc. and Monsanto Company as a result of activities undertaken within the scope of a joint research agreement in effect on or before the date the claimed invention was made.

### FIELD OF THE INVENTION

The present invention relates to plant genomics and plant improvement

### BACKGROUND OF THE INVENTION

Beneath the cuticle epidermis, tomato fruit have a fleshy pericarp that consists of highly vacuolated cells, similar to leaf palisade cells. In young fruit, the pericarp cells contain photosynthetically active chloroplasts which, as the fruit develop, undergo a transition to chromoplasts that no longer fix carbon (Smillie et al., 1999; Piechulla et al., 1987; Blanke and Lenz, 1989; Gillaspy et al., 1993). Most of the photosynthate accumulation in fruit comes from photosynthesis in leaves, although it has been estimated that a small portion, 10-15%, of the total carbon in tomato fruit results from the fruit's photosynthetic activity (Whiley et al., 1992; Marcelis and Baan Hofman-Eijer, 1995; Hetherington et al., 1998). Dark adapted fruit are nearly as photosynthetically efficient as leaves (Hetherington et al., 1998) and the proteins involved in light harvesting electron transfer and CO₂ fixation are present in fruit (Carrara et al., 2001).

In young developing tomato fruit, the expression of chloroplast photosynthetic proteins is similar to that in leaves, but differences have been observed that suggest that some regulation of photosynthesis may be fruit specific. For example, only two of the five ribulose-1,5 bisphosphate carboxylase (rbcS) identified in leaves are expressed in developing fruit (Sugita and Gruissem, 1987; Wanner and Gruissem, 1991). Some of the fruit-specific transcriptional regulation of photosynthetic functions may be a result of the sink state of the fruit (Manzara et al., 1993) but are also regulated by fruit development and ripening (Simpson et al., 1976). Young tomato fruit contain chloroplasts with chlorophyll but as the fruit ripen, chlorophyll a is degraded by chlorophyllase and a multi-step decomposition pathway. While many aspects of fruit development are known, how fruit development and ripening regulate the function and inactivation of photosynthetically active chloroplasts in fruit is not well understood. Transcription factors modify the expression of sets of genes through binding to specific DNA sequences and other regulatory proteins. Often transcription factors modify the expression of suites of genes involved in complex processes and may function as precise modulators of processes with multiple inputs. The developmental and ripening programs of fruit and the environment in which the fruit is localized potentially influence fruit photosynthetic activity, suggesting that fruit chloroplast biogenesis and metabolism may be responsive to multiple inputs and potential sites of regulation. Chloroplast degradation in ripening fruit apparently is at least partially regulated by the transcription factors, Rin and Nor, since mutations in these genes result in fruit that do not ripen and remain green with repressed chlorophyll degradation (Giovannoni, 2007).

Sequencing the Arabidopsis genome identified approximately 1700 transcription factors (Riechmann et al., 2000; Riechmann and Ratcliffe, 2000). The functions of some of these transcription factors have been inferred by examining the phenotypes of Arabidopsis lines with mutations that eliminate or alter the function of specific transcription factors, but phenotypes that relate to fleshy fruit development and morphology may not be obvious from studies utilizing Arabidopsis. In tomato, the genome sequence is not complete and consequently it is not possible to identify a complete set of transcription factors. By expressing Arabidopsis transcription factors in tomato and analyzing the consequences for the fruit structure and physiology, changes may be observed that suggest heretofore unrevealed functions for the Arabidopsis transcription factors and also predict potential homologous or interacting tomato proteins.

### SUMMARY OF THE INVENTION

The present invention pertains to transgenic plants, and methods for producing such transgenic plants, where the transgenic plant comprises a stably integrated, recombinant polynucleotide, for example, a nucleic acid construct, that comprises a constitutive or plant organ-associated promoter and a nucleic acid sequence that encodes a transcription factor polypeptide. The promoter is functional in plant cells and regulates transcription of the nucleic acid sequence, and may be either a constitutive or organ-enhanced promoter (e.g., a fruit-enhanced promoter). The polypeptide is a member of the GARP family of transcription factors, and the polypeptide has an amino acid percent identity with any of SEQ ID NOs: 2, 4, 6, 8, 10, 12, 14, 16 or 18, or domains SEQ ID NO: 19-36, or consensus sequences SEQ ID NOs: 43 or 44, said amino acid percentage identities and degrees of similarity described below. The transgenic plant is selected from a population of transgenic plants that comprise the recombinant polynucleotide, said selection performed by screening the population of transgenic plants that express the polypeptide for an enhanced trait. in a plant organ relative to an analogous plant organ in a control plant that does not have the recombinant polynucleotide. The enhanced trait may include earlier chloroplast development, darker green color when grown or maintained in the absence of light, larger chloroplasts, more extensive chloroplast thylakoid granal development, elevated carbohydrate levels, or elevated chlorophyll levels. The carbohydrate may be a sugar or starch, and the plant organ may include leaves, fruit, roots, seeds, stems, or flower parts. The transgenic plant may be a tomato plant or any other plant species.

### BRIEF DESCRIPTION OF THE SEQUENCE LISTING AND DRAWINGS

The Sequence Listing provides exemplary polynucleotide and polypeptide sequences of the invention. The traits associated with the use of the sequences are included in the Examples.

Incorporation of the Sequence Listing. The copy of the Sequence Listing, being submitted electronically with this patent application, provided under 37 CFR §1.821-1.825, is a read-only memory computer-readable file in ASCII text format. The Sequence Listing is named “MBI-0086P_ST25.txt”, the electronic file of the Sequence Listing was created on Dec. 9, 2008, and is 73,744 bytes in size, or 73 kilobytes in size as measured in MS-WINDOWS. The Sequence Listing is herein incorporated by reference in its entirety.

FIG. 1: morphology of fruit from AtGLK1 and AtGLK2 expressing lines. Immature, mature green and red ripe fruit from control (FIG. 1A) and transgenic lines expressing AtGLK1 (FIGS. 1B, 1D, 1F, and 1H) or AtGLK2 (FIGS. 1C, 1E, 1G, and 1I) with the 35S (FIGS. 1B and 1C), LTP (FIGS. 1D and 1E), RbcS (FIGS. 1F and 1G) or phytoene desaturase (PD; FIGS. 1H and 1I) promoters. From left to right fruit were 6, 18, 25, 32, 39 days after anthesis and the red fruit are representative of turning and fully red ripe stages.

FIG. 2: morphology of very young fruit (1 to 8 days after anthesis) from lines containing the LTP (FIG. 2A) or RbcS (FIG. 2B) promoter expressing AtGLK1 (middle column) or AtGLK2 (right column). Control fruit are shown on the left in each panel.

FIGS. 3, 4 and 5: chlorophyll in mature green fruit and lycopene from red ripe fruit from AtGLK1 and AtGLK2 expressing lines. Chlorophyll extracted from pericarp of mature green fruit (FIGS. 3A and 3B) and from leaves (FIGS. 4A and 4B) was measured spectrophotometrically. The amount of chlorophyll was calculated using [chl a mg/L]=12.7×Abs.₆₃₃−2.69×Abs.₆₄₅ and [chl b mg/L]=22.9×Abs.₆₄₅−4.8×Abs.₆₃₃ (Arnon, 1949). Lycopene (FIGS. 5A and 5B) from red ripe fruit was measured spectrophotometrically (510 nm). Fruit and leaves were from AtGLK1 (FIGS. 3A, 4A, and 5A) or AtGLK2 (FIGS. 3B, 4B, and 5B) expressing plants. Results shown are for fruit from plants grown in greenhouses.

FIG. 6: chloroplast morphology in lines expressing AtGLK1 and AtGLK2 by the 35S promoter. Typical chloroplasts were observed in sections of immature (FIGS. 6A, 6E, and 6I) and mature green (FIGS. 6B, 6F, and 6J) and chromoplasts in red ripe fruit (FIGS. 6C, 6G, and 6K) expressing AtGLK1 (FIGS. 6A, 6B, 6C, and 6D), AtGLK2 (FIGS. 6E, 6F, 6G, and 6H) and control fruit (FIGS. 6I, 6J, 6K, and 6L) fixed and examined by transmission electron microscopy. Chloroplasts from fully expended leaves of AtGLK1 (FIG. 6D), AtGLK2 (FIG. 6H) expressing and control plants (FIG. 6L) are shown. A 1 μm scale bar is shown.

FIG. 7: starch content of mature green fruit from 35S:AtGLK1, 355:AtGLK2 expressing and control lines.

FIG. 8: staining for starch in fresh cut sections of green fruit. Hand cut sections of green fruit with diameters of 1 cm (FIGS. 8A, 8D, and 8G, immature green, about seven days post anthesis), 2.5 cm (FIGS. 8B, 8E, and 8H, 14 days post anthesis), or mature green fruit (FIGS. 8C, 8F, and 8I) from control (FIGS. 8A, 8B, and 8C), 35S:AtGLK1 (FIGS. 8D, 8E, and 8F), or 35S:AtGLK2 (FIGS. 8G, 8H, and 8I) plants.

FIG. 9: BRIX measurements of red ripe fruit from AtGLK1 (FIG. 9A) and AtGLK2 (FIG. 9B) expressing lines and total neutral sugars (FIG. 9C). Total neutral sugars were measured for 35S:AtGLK1, 35S:AtGLK2 expressing and control lines.

FIG. 10: appearance of green fruit that developed in the absence of light and harvested 35 days after anthesis. Top row: fruit which had developed in normal light conditions. Bottom row: fruit which had been placed in light-blocking bags shortly after anthesis. Left: Control fruit. Right: fruit expressing AtGLK1:35S.

FIG. 11: alignments of the Myb-like DNA binding domains and GCT domains of AtGLK1, AtGLK2, and phylogenetically related sequences, are shown in this figure. Below each alignment are consensus sequences for the Myb-like DNA binding domains and GCT domains, SEQ ID NO: 43 and 44, respectively. SEQ ID NOs: appear in parentheses.

### DETAILED DESCRIPTION OF THE INVENTION

The present invention relates to polynucleotides and polypeptides for modifying phenotypes of plants, particularly those associated with altered carbohydrate or chlorophyll content in plants and plant organs. Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a trait” is a reference to one or more traits and equivalents thereof known to those skilled in the art, and so forth.

### DEFINITIONS

“Polynucleotide” is a nucleic acid molecule comprising a plurality of polymerized nucleotides, e.g., at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and that may be used to determine the limits of the genetically active unit (Rieger et al. (1976)). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues. In many instances, a polypeptide comprises a polymerized amino acid residue sequence that is a transcription factor or a domain or portion or fragment thereof. Additionally, the polypeptide may comprise: (i) a localization domain; (ii) an activation domain; (iii) a repression domain; (iv) an oligomerization domain; (v) a DNA-binding domain; or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical, matching or corresponding nucleotides at positions shared by the polynucleotide sequences. A degree of identity of polypeptide sequences is a function of the number of identical amino acids at corresponding positions shared by the polypeptide sequences. A degree of homology or similarity of polypeptide sequences is a function of the number of amino acids at corresponding positions shared by the polypeptide sequences.

“Alignment” refers to a number of nucleotide bases or amino acid residue sequences aligned by lengthwise comparison so that components in common (i.e., nucleotide bases or amino acid residues at corresponding positions) may be visually and readily identified. The fraction or percentage of components in common is related to the homology or identity between the sequences. An alignment of phylogenetically-related sequences may be used to identify conserved domains and relatedness within these domains. An alignment may suitably be determined by means of computer programs known in the art such as MACVECTOR software (1999) (Accelrys, Inc., San Diego, Calif.) or ClustalX© (Larkin et al., 2007). The latter is available at www.clustal.org.

Two or more sequences may be “optimally aligned” with a similarity scoring method using a defined amino acid substitution matrix such as the BLOSUM62 scoring matrix. The preferred method uses a gap existence penalty and gap extension penalty that arrives at the highest possible score for a given pair of sequences. See, for example, Dayhoff et al. (1978) and Henikoff and Henikoff (1992). The BLOSUM62 matrix is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. Optimal alignment may be accomplished manually or with a computer-based alignment algorithm, such as gapped BLAST 2.0 (Altschul et al, (1997); or at www.ncbi.nlm.nih.gov. See U.S. Patent Application US20070004912.

A “conserved domain” or “conserved region” as used herein refers to a region in heterologous polynucleotide or polypeptide sequences where there is a relatively high degree of sequence identity between the distinct sequences. For example, a “Myb-like domain”, a putative DNA binding domain, is found in a polypeptide member of GARP transcription factor family and is an example of a conserved domain. With respect to polynucleotides encoding presently disclosed transcription factors, a conserved domain is preferably at least nine base pairs (bp) in length. Sequences that possess or encode for conserved domains that meet these criteria of percentage identity, and that have comparable biological activity to the present transcription factor sequences, thus being members of a clade of transcription factor polypeptides, are encompassed by the invention. A fragment or domain can be referred to as outside a conserved domain, outside a consensus sequence, or outside a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

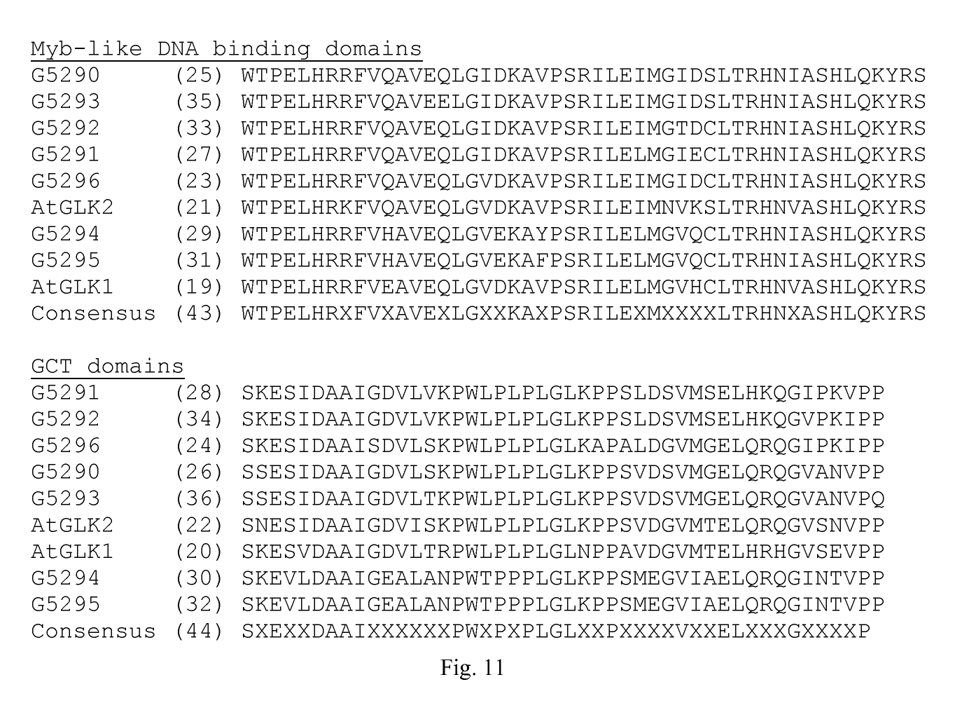

As one of ordinary skill in the art recognizes, conserved domains may be identified as regions or domains of identity to a specific consensus sequence (see, for example, Riechmann et al. (2000), Riechmann and Ratcliffe (2000)). Thus, by using alignment methods well known in the art, the conserved domains of the plant transcription factors, for example, for the GARP proteins, may be determined. Conserved domains determined by such methods are shown in FIG. 11.

The conserved domains for many of the transcription factor sequences of the invention are listed in Tables 1b and 2b. Also, the polypeptides of Tables 1a, 1b, 2a and 2b have conserved domains specifically indicated by amino acid coordinate start and stop sites. A comparison of the regions of these polypeptides allows one of skill in the art to identify domains or conserved domains for any of the polypeptides listed or referred to in this disclosure.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985), Sambrook et al. (1989), and by Haymes et al. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, encoded transcription factors having 38% or greater identity with the conserved domain of disclosed transcription factors.

The terms “paralog” and “ortholog” are defined below in the section entitled “Orthologs and Paralogs”. In brief, orthologs and paralogs are evolutionarily related genes that have similar sequences and functions. Orthologs are structurally related genes in different species that are derived by a speciation event. Paralogs are structurally related genes within a single species that are derived by a duplication event.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “tigr.org” under the heading “Terms associated with TIGRFAMs”.

In general, the term “variant” refers to molecules with some differences, generated synthetically or naturally, in their base or amino acid sequences as compared to a reference (native) polynucleotide or polypeptide, respectively. These differences include substitutions, insertions, deletions or any desired combinations of such changes in a native polynucleotide of amino acid sequence.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations may result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a transcription factor nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor. Thus, it will be readily appreciated by those of skill in the art, that any of a variety of polynucleotide sequences is capable of encoding the transcription factors and transcription factor homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as a significant amount of the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, e.g., replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (see U.S. Pat. No. 5,840,544).

The invention also encompasses production of DNA sequences that encode transcription factors and transcription factor derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding transcription factors or any fragment thereof.

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae.

A “control plant” as used in the present invention refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain a nucleic acid construct such as an expression vector or cassette. The expression cassette typically comprises a polypeptide-encoding sequence operably linked (i.e., under regulatory control of) to appropriate inducible or constitutive regulatory sequences that allow for the controlled expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, including transgenic seed, fruit, leaf, or root, plant tissue, plant cells or any other transgenic plant material, e.g., a transformed plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a transcription factor expression is altered, e.g., in that it has been knocked out, overexpressed, or ectopically expressed.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as morphological analysis. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease, or an even greater difference, in an observed trait as compared with a control or wild-type plant. It is known that there can be a natural variation in the modified trait. Therefore, the trait modification observed entails a change of the normal distribution and magnitude of the trait in the plants as compared to control or wild-type plants.

“Ectopic expression or altered expression” in reference to a polynucleotide indicates that the pattern of expression in, e.g., a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression of that gene in a wild-type plant, cell or tissue, at any developmental or temporal stage. Overexpression can occur when, for example, the genes encoding one or more transcription factors are under the control of a regulatory control element such as a strong or constitutive promoter (e.g., the cauliflower mosaic virus 35S transcription initiation region). Overexpression may also be achieved by placing a gene of interest under the control of an inducible or tissue specific promoter, or may be achieved through integration of transposons or engineered T-DNA molecules into regulatory regions of a target gene. Thus, overexpression may occur throughout a plant, in specific tissues of the plant, or in the presence or absence of particular environmental signals, depending on the promoter or overexpression approach used.

Overexpression may take place in plant cells normally lacking expression of polypeptides functionally equivalent or identical to the present transcription factors. Overexpression may also occur in plant cells where endogenous expression of the present transcription factors or functionally equivalent molecules normally occurs, but such normal expression is at a lower level. Overexpression thus results in a greater than normal production, or “overproduction” of the transcription factor in the plant, cell or tissue.

In addition to the use of constitutive promoters, overexpression may also be regulated by tissue-enhanced or associated promoters such as, for example, organ-enhanced or organ-associated promoters, or specifically fruit-associated promoters. As used herein, the term “tissue-associated promoter” refers to any promoter that directs RNA synthesis at a higher level in a particular type of cell and/or tissue (for example, a fruit-associated promoter).

As used herein, “low light” refers to a light intensity ranging from 0.001 to 10 μmoles/m²/sec.

Transcription Factors Modify Expression of Endogenous Genes

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding site or DNA-binding site motif (see, for example, Riechmann et al. (2000a)). The plant transcription factors of the present invention belong to particular transcription factor families indicated in the Sequence Listing and in the Tables found herein.

Generally, the transcription factors encoded by the present sequences are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with enhanced traits. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from any source whether natural, synthetic, semi-synthetic or recombinant. The sequences of the invention may also include fragments of the present amino acid sequences. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, e.g., mutation reactions, PCR reactions, or the like; as substrates for cloning e.g., including digestion or ligation reactions; and for identifying exogenous or endogenous modulators of the transcription factors. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) and Peng et al. (1999). In addition, many others have demonstrated that an Arabidopsis transcription factor expressed in an exogenous plant species elicits the same or very similar phenotypic response. See, for example, Fu et al. (2001); Nandi et al. (2000); Coupland (1995); and Weigel and Nilsson (1995)).

In another example, Mandel et al. (1992), and Suzuki et al. (2001), teach that a transcription factor expressed in another plant species elicits the same or very similar phenotypic response of the endogenous sequence, as often predicted in earlier studies of Arabidopsis transcription factors in Arabidopsis (see Mandel et al. (1992); Suzuki et al. (2001)). Other examples include Müller et al. (2001); Kim et al. (2001); Kyozuka and Shimamoto (2002); Boss and Thomas (2002); He et al. (2000); and Robson et al. (2001).

In yet another example, Gilmour et al. (1998) teach an Arabidopsis AP2 transcription factor, CBF1, which, when overexpressed in transgenic plants, increases plant freezing tolerance. Jaglo et al. (2001) further identified sequences in Brassica napus which encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues which bracket the AP2/EREBP DNA binding domains of the proteins and distinguish them from other members of the AP2/EREBP protein family. (Jaglo et al. (2001))

Transcription factors mediate cellular responses and control traits through altered expression of genes containing cis-acting nucleotide sequences that are targets of the introduced transcription factor. It is well appreciated in the art that the effect of a transcription factor on cellular responses or a cellular trait is determined by the particular genes whose expression is either directly or indirectly (e.g., by a cascade of transcription factor binding events and transcriptional changes) altered by transcription factor binding. In a global analysis of transcription comparing a standard condition with one in which a transcription factor is overexpressed, the resulting transcript profile associated with transcription factor overexpression is related to the trait or cellular process controlled by that transcription factor. For example, the PAP2 gene and other genes in the MYB family have been shown to control anthocyanin biosynthesis through regulation of the expression of genes known to be involved in the anthocyanin biosynthetic pathway (Bruce et al. (2000); and Borevitz et al. (2000)). Further, global transcript profiles have been used successfully as diagnostic tools for specific cellular states (e.g., cancerous vs. non-cancerous; Bhattacharjee et al. (2001); and Xu et al. (2001)). Consequently, it is evident to one skilled in the art that similarity of transcript profile upon overexpression of different transcription factors would indicate similarity of transcription factor function.

Polypeptides and Polynucleotides of the Invention

The present invention provides, among other things, transcription factors (TFs), and transcription factor homolog polypeptides, and isolated or recombinant polynucleotides encoding the polypeptides, or novel sequence variant polypeptides or polynucleotides encoding novel variants of transcription factors derived from the specific sequences provided in the Sequence Listing. Also provided are methods for enhancing a plant traits, for example, earlier chloroplast development, darker green color when an organ such as fruit is developed in the absence of light, larger chloroplasts, more extensive chloroplast thylakoid granal development, more carbohydrate, or more chlorophyll.

These methods are based on the ability to alter the expression of critical regulatory molecules that may be conserved between diverse plant species. Related conserved regulatory molecules may be originally discovered in a model system such as Arabidopsis and homologous, functional molecules then discovered in other plant species. The latter may then be used to confer enhanced traits of the invention in diverse plant species.

Exemplary polynucleotides encoding the polypeptides of the invention were identified in the Arabidopsis thaliana GenBank database using publicly available sequence analysis programs and parameters. Sequences initially identified were then further characterized to identify sequences comprising specified sequence strings corresponding to sequence motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including full length coding sequences, were subsequently recovered by the rapid amplification of cDNA ends (RACE) procedure using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

The sequences in the Sequence Listing, derived from diverse plant species, may be ectopically expressed in overexpressor plants. The changes in the characteristic(s) or trait(s) of the plants are then observed and found to confer the enhanced traits of the present invention. Therefore, the polynucleotides and polypeptides can be used to improve desirable characteristics of plants.

The polynucleotides of the invention may also be ectopically expressed in overexpressor plant cells and the changes in the expression levels of a number of genes, polynucleotides, and/or proteins of the plant cells observed. Therefore, the polynucleotides and polypeptides can be used to change expression levels of a genes, polynucleotides, and/or proteins of plants or plant cells.

The data presented herein represent the results obtained in experiments with transcription factor polynucleotides and polypeptides that may be expressed in plants for the purpose of enhancing plant traits such as earlier chloroplast development, darker green color when developed in the absence of light, larger chloroplasts, more extensive chloroplast thylakoid granal development, more carbohydrate, and more chlorophyll.

Expression of GARP Family Transcription Factor Enhances Valuable Traits in Plant Organs

Transcription factors that control fruit chloroplast development were identified by surveying fruit phenotypes in tomato lines transgenically expressing Arabidopsis transcription factors.

Analysis of a population of transgenic tomato lines expressing over 1000 Arabidopsis transcription factors revealed for that the expression of two transcription factors profoundly influenced fruit green color and the chloroplast morphology in developing unripe tomato fruit. The two transcription factors effecting green tomato fruit were members of the GARP transcription factor family, AtGLK1 (golden2-like protein 1, NCBI accession no. AAK20120; SEQ ID NO: 2) and AtGLK2 (golden2-like protein 1, NCBI accession no. AAK20121; SEQ ID NO: 4) (Fitter et al., 2002). Expression of AtGLK1 or AtGLK2 resulted in darker green tomato fruit, green fruit chloroplasts with significantly altered thylakoid granal structures and greater green fruit starch accumulation and ultimately increased sugar accumulation in ripe fruit. While observations of Arabidopsis mutants of AtGlk1 and AtGlk2 and the double AtGlk1/2 mutant (Fitter et al., 2002) suggested that these transcription factors are important in chloroplast development and structure, only by expressing these two transcription factors in a species like tomato was it possible to see the significance of their expression for carbohydrate levels and the effects of dark treatments in fleshy fruit.

The GLK pair of monophyletic nuclear GARP transcription factors regulate chloroplast biogenesis and maintenance in maize, rice and Arabidopsis (Fitter et al., 2002). In Arabidopsis AtGLK1 and AtGLK2 appear to act redundantly and cell autonomously (Waters et al., 2008). Fitter et al., (2002) have suggested that because these GLK transcription factors are not found in cyanobacteria, these transcription factors are necessary for chloroplast assembly and not photosynthesis. The GLK transcription factors in maize, rice, Arabidopsis, and the moss Physcomitrella patens form a monophyletic clade (Fitter et al., 2002; Yasumura et al., 2005). Genes in this clade contain both the myb-like DNA binding domain typical of GARP family transcription factors (Riechmann et al., 2000), and a second C-terminal conserved domain known as the GCT domain (Rossini et al., 2001; Yasumura et al., 2005).

The GLK transcription factors are crucial for chloroplast development in C3 and C4 photosynthetic leaf tissues in maize, and in leaf chloroplast development in rice and Arabidopsis. Transposon mutants in the maize GLK transcription factor, ZmGLK2, have smaller, less granal chloroplasts in both the C3 and C4 tissues; the leaf blades were pale green and the bundle sheath was white. These mutations perturb chloroplast development in the bundle sheath cells independent of light but do not effect rbcS accumulation (Hall et al., 1998; Cribb et al., 2001). A ZmGLK2 homologue was identified, ZmGLK1, that is regulated by light and participates in chloroplast biogenesis in C4 mesophyll tissues. In the C3 plant, Arabidopsis, the GLK homologues, AtGLK1 and AtGLK2, are largely redundant (Waters et al., 2008). AtGLK1 and AtGLK2 are expressed in photosynthesizing tissues and some accumulation of AtGLK2 has been observed in roots and siliques. AtGLK1 expression is expressed in response to light and AtGLK2 is apparently regulated by circadian and light-induced mechanisms (Fitter et al., 2002). AtGLK2 probably functions in the conversion of etioplasts to chloroplasts. Double mutants in AtGLK1 and AtGLK2 have noticeably lighter leaves and chloroplasts lacking granal thylakoid membranes and at least some of the proteins associated with photosystem II (PSII) (Fitter et al., 2002). Partial complementation of the Arabidopsis AtGLK1-AtGLK2 double mutant by the moss Physcomitrella patens PpGLK1 suggests that GLKs are functionally similar in both bryophytes and vascular plants (Yasumura et al., 2005). The promoter regions of some chlorophyll biosynthetic enzymes and some of the light harvesting complex proteins (LHCP1 and LHCP6) have multiple copies of the 5 by sequence that is the target of other GARP ARR-B transcription factions.

As AtGLK1 and AtGLK2 apparently interact, they also may be capable of interacting with GLK homologues in tomato. AtGLK1 is probably most similar to the tomato sequence SGN-U226143 (52% aa) that has been identified in flower libraries and AtGLK2 is most similar to SGN-U231251 (56% aa), that has been identified in leaf and flower libraries. A third GLK-like sequence also exists in tomato. Other expression data for these tomato homologues is not currently available. AtGLK1 and AtGLK2 sequences are about 45% similar. Expression of AtGLK1 and AtGLK2 in tomato suggests that the homologous tomato transcription factors may be important for chloroplast biogenesis and structure in green fruit.

The constitutive expression of either AtGLK1 or AtGLK2 changes chlorophyll abundance in green fruit. Expression of AtGLK1 also promotes the formation of chloroplasts at very early stages in fruit development. Manipulation of the endogenous tomato GLK homologues may reveal further functions of this class of transcription factors.

Changes in the chloroplasts in green fruit as a consequence of AtGLK1 or AtGLK2 expression result in green fruit that accumulate more starch than control fruit. Increased BRIX values and sugars were observed in the red fruit in lines expressing AtGLK1, although light conditions may influence how much the transcription factor expression contributes to these phenotypes.

Unexpectedly, when fruit expressing AtGLK1 developed in the absence of light, the fruit were noticeably greener than control fruit that developed in similar light-blocking conditions. These novel results indicate that proteins with AtGLK1 function can act to promote and/or maintain chloroplast development and chlorophyll levels in plant organs in the absence of light or in low light levels. As such, these transcription factors are expected to be useful in enhancing the appearance, photosynthetic capacity, and carbohydrate levels in plant organs (e.g. leaves, roots, fruits, seeds) under low light or dark conditions.

### Orthologs and Paralogs

Homologous sequences as described above can comprise orthologous or paralogous sequences. Several different methods are known by those of skill in the art for identifying and defining these functionally homologous sequences. General methods for identifying orthologs and paralogs, including phylogenetic methods, sequence similarity and hybridization methods, are described herein; an ortholog or paralog, including equivalogs, may be identified by one or more of the methods described below.

As described by Eisen (1998), evolutionary information may be used to predict gene function. It is common for groups of genes that are homologous in sequence to have diverse, although usually related, functions. However, in many cases, the identification of homologs is not sufficient to make specific predictions because not all homologs have the same function. Thus, an initial analysis of functional relatedness based on sequence similarity alone may not provide one with a means to determine where similarity ends and functional relatedness begins. Fortunately, it is well known in the art that protein function can be classified using phylogenetic analysis of gene trees combined with the corresponding species. Functional predictions can be greatly improved by focusing on how the genes became similar in sequence (i.e., by evolutionary processes) rather than on the sequence similarity itself (Eisen, 1998). In fact, many specific examples exist in which gene function has been shown to correlate well with gene phylogeny (Eisen, 1998). Thus, “[t]he first step in making functional predictions is the generation of a phylogenetic tree representing the evolutionary history of the gene of interest and its homologs. Such trees are distinct from clusters and other means of characterizing sequence similarity because they are inferred by techniques that help convert patterns of similarity into evolutionary relationships . . . . After the gene tree is inferred, biologically determined functions of the various homologs are overlaid onto the tree. Finally, the structure of the tree and the relative phylogenetic positions of genes of different functions are used to trace the history of functional changes, which is then used to predict functions of [as yet] uncharacterized genes” (Eisen, 1998).

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al., 1994; Higgins et al., 1996). Groups of similar genes can also be identified with pair-wise BLAST analysis (Feng and Doolittle, 1987). For example, a clade of very similar MADS domain transcription factors from Arabidopsis all share a common function in flowering time (Ratcliffe et al., 2001), and a group of very similar AP2 domain transcription factors from Arabidopsis are involved in tolerance of plants to freezing (Gilmour et al., 1998). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount, 2001).

Transcription factor gene sequences are conserved across diverse eukaryotic species lines (Goodrich et al., 1993; Lin et al., 1991; Sadowski et al., 1988). Plants are no exception to this observation; diverse plant species possess transcription factors that have similar sequences and functions. Speciation, the production of new species from a parental species, gives rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al., 1994); Higgins et al., 1996) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

By using a phylogenetic analysis, one skilled in the art would recognize that the ability to predict similar functions conferred by closely-related polypeptides is predictable. This predictability has been confirmed by our own many studies in which we have found that a wide variety of polypeptides have orthologous or closely-related homologous sequences that function as does the first, closely-related reference sequence. For example, distinct transcription factors, including:

(i) AP2 family Arabidopsis G47 (found in U.S. Pat. No. 7,135,616, issued 14 Nov. 2006), a phylogenetically-related sequence from soybean, and two phylogenetically-related homologs from rice all can confer greater tolerance to drought, hyperosmotic stress, or delayed flowering as compared to control plants;

(ii) CAAT family Arabidopsis G481 (found in PCT patent publication WO2004076638), and numerous phylogenetically-related sequences from dicots and monocots can confer greater tolerance to drought-related stress as compared to control plants;

(iii) Myb-related Arabidopsis G682 (found in U.S. Pat. No. 7,223,904, issued 29 May 2007) and numerous phylogenetically-related sequences from dicots and monocots can confer greater tolerance to heat, drought-related stress, cold, and salt as compared to control plants;

(iv) WRKY family Arabidopsis G1274 (found in U.S. Pat. No. 7,196,245, issued 27 Mar. 2007) and numerous closely-related sequences from dicots and monocots have been shown to confer increased water deprivation tolerance, and

(v) AT-hook family soy sequence G3456 (found in US patent publication 20040128712A1) and numerous phylogenetically-related sequences from dicots and monocots, increased biomass compared to control plants when these sequences are overexpressed in plants.

The polypeptides sequences in the above-listed patent publications belong to distinct clades of polypeptides that include members from diverse species. In each case, most or all of the clade member sequences derived from both dicots and monocots have been shown to confer increased tolerance to one or more abiotic stresses when the sequences were overexpressed, and hence will likely increase yield and or crop quality. These studies each demonstrate that evolutionarily conserved genes from diverse species are likely to function similarly (i.e., by regulating similar target sequences and controlling the same traits), and that polynucleotides from one species may be transformed into closely-related or distantly-related plant species to confer or enhance traits.

At the nucleotide level, the claimed sequences will typically share at least about 30% or 40% nucleotide sequence identity, preferably at least about 50%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, or at least about 80% sequence identity, and more preferably at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more sequence identity, or about 100% sequence identity, to one or more of the listed full-length sequences such as SEQ ID NO: 1, 3, 5, 7, 9, 11, 13, 15, or 17, or to a region of a listed sequence excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

At the polypeptide level, the sequences of the invention will typically share, including conservative substitutions, at least 29%, or at least 30%, or at least 32%, or at least 33%, or at least 38%, or at least 41%, or at least 42%, or at least 43%, or at least 44%, or at least 46%, or at least 47%, or at least 55%, or at least 56%, or at least 57%, or at least 58%, or at least 59%, or at least 60%, or at least 61%, or at least 62% sequence identity, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, or 100% amino acid residue sequence identity, to one or more of the listed full-length sequences such as SEQ ID NO: 2, 4, 6, 8, 10, 12, 14, 16, or 18, or to a listed sequence but excluding or outside of the known consensus sequence or consensus DNA-binding site.

A conserved domain with respect to presently disclosed polypeptides refers to a domain within a transcription factor family that exhibits a higher degree of sequence homology, such as at least about 38% amino acid sequence identity including conservative substitutions, or at least about 42% sequence identity, or at least about 45% sequence identity, or at least about 48% sequence identity, or at least about 50% sequence identity, or at least about 51% sequence identity, or at least about 52% sequence identity, or at least about 53% sequence identity, or at least about 54% sequence identity, or at least about 55% sequence identity, or at least about 56% sequence identity, or at least about 57% sequence identity, or at least about 58% sequence identity, or at least about 59% sequence identity, or at least about 60% sequence identity, or at least about 61% sequence identity, or at least about 62% sequence identity, or at least about 63% sequence identity, or at least about 64% sequence identity, or at least about 65% sequence identity, or at least about 66% sequence identity, or at least about 67% sequence identity, or at least about 68% sequence identity, or at least about 69% sequence identity, or at least about 70% sequence identity, or at least about 71% sequence identity, or at least about 72% sequence identity, or at least about 73% sequence identity, or at least about 74% sequence identity, or at least about 75% sequence identity, or at least about 76% sequence identity, or at least about 77% sequence identity, or at least about 78% sequence identity, or at least about 79% sequence identity, or at least about 80% sequence identity, or at least about 81% sequence identity, or at least about 82% sequence identity, or at least about 83% sequence identity, or at least about 84% sequence identity, or at least about 85% sequence identity, or at least about 86% sequence identity, or at least about 87% sequence identity, or at least about 88% sequence identity, or at least about 89% sequence identity, or at least about 90% sequence identity, or at least about 91% sequence identity, or at least about 92% sequence identity, or at least about 93% sequence identity, or at least about 94% sequence identity, or at least about 95% sequence identity, or at least about 96% sequence identity, or at least about 97% sequence identity, or at least about 98% sequence identity, or at least about 99% sequence identity, or 100% amino acid residue sequence identity, to a conserved domain of a polypeptide of the invention, such as those listed in the present tables or Sequence Listing (e.g., SEQ ID NO: 19-36, or consensus sequences 43 or 44).

Percent identity can be determined electronically, e.g., by using the MEGALIGN program (DNASTAR, Inc. Madison, Wis.). The MEGALIGN program can create alignments between two or more sequences according to different methods, for example, the clustal method (see, for example, Higgins and Sharp, 1988). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (see U.S. Pat. No. 6,262,333).

Software for performing BLAST analyses is publicly available, e.g., through the National Center for Biotechnology Information (see internet website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, 1990; Altschul et al., 1993). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, n=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1992). Unless otherwise indicated for comparisons of predicted polynucleotides, “sequence identity” refers to the % sequence identity generated from a tblastx using the NCBI version of the algorithm at the default settings using gapped alignments with the filter “off” (see, for example, internet website at www.ncbi.nlm.nih.gov/).

Other techniques for alignment are described by Doolittle, 1996. Preferably, an alignment program that permits gaps in the sequence is utilized to align the sequences. The Smith-Waterman is one type of algorithm that permits gaps in sequence alignments (see Shpaer, 1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (see, for example, Hein, 1990) Identity between sequences can also be determined by other methods known in the art, e.g., by varying hybridization conditions (see US Patent Application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al., 1997), PFAM, and other databases which contain previously identified and annotated motifs, sequences and gene functions. Methods that search for primary sequence patterns with secondary structure gap penalties (Smith et al., 1992) as well as algorithms such as Basic Local Alignment Search Tool (BLAST; Altschul, 1990; Altschul et al., 1993), BLOCKS (Henikoff and Henikoff, 1991), Hidden Markov Models (HMM; Eddy, 1996; Sonnhammer et al., 1997), and the like, can be used to manipulate and analyze polynucleotide and polypeptide sequences encoded by polynucleotides. These databases, algorithms and other methods are well known in the art and are described in Ausubel et al., 1997, and in Meyers, 1995.

A further method for identifying or confirming that specific homologous sequences control the same function is by comparison of the transcript profile(s) obtained upon overexpression or knockout of two or more related polypeptides. Since transcript profiles are diagnostic for specific cellular states, one skilled in the art will appreciate that genes that have a highly similar transcript profile (e.g., with greater than 50% regulated transcripts in common, or with greater than 70% regulated transcripts in common, or with greater than 90% regulated transcripts in common) will have highly similar functions. Fowler and Thomashow (2002), have shown that three paralogous AP2 family genes (CBF1, CBF2 and CBF3) are induced upon cold treatment, and each of which can condition improved freezing tolerance, and all have highly similar transcript profiles. Once a polypeptide has been shown to provide a specific function, its transcript profile becomes a diagnostic tool to determine whether paralogs or orthologs have the same function.

Furthermore, methods using manual alignment of sequences similar or homologous to one or more polynucleotide sequences or one or more polypeptides encoded by the polynucleotide sequences may be used to identify regions of similarity and conserved domains characteristic of a particular transcription factor family. Such manual methods are well-known of those of skill in the art and can include, for example, comparisons of tertiary structure between a polypeptide sequence encoded by a polynucleotide that comprises a known function and a polypeptide sequence encoded by a polynucleotide sequence that has a function not yet determined. Such examples of tertiary structure may comprise predicted α-helices, β-sheets, amphipathic helices, leucine zipper motifs, zinc finger motifs, proline-rich regions, cysteine repeat motifs, and the like.

Orthologs and paralogs of presently disclosed polypeptides may be cloned using compositions provided by the present invention according to methods well known in the art. cDNAs can be cloned using mRNA from a plant cell or tissue that expresses one of the present sequences. Appropriate mRNA sources may be identified by interrogating Northern blots with probes designed from the present sequences, after which a library is prepared from the mRNA obtained from a positive cell or tissue. Polypeptide-encoding cDNA is then isolated using, for example, PCR, using primers designed from a presently disclosed gene sequence, or by probing with a partial or complete cDNA or with one or more sets of degenerate probes based on the disclosed sequences. The cDNA library may be used to transform plant cells. Expression of the cDNAs of interest is detected using, for example, microarrays, Northern blots, quantitative PCR, or any other technique for monitoring changes in expression. Genomic clones may be isolated using similar techniques to those.

Examples of orthologs of the Arabidopsis polypeptide sequences and their functionally similar orthologs are listed in Tables 1a, 1b, 2a and 2b and the Sequence Listings, and include Arabidopsis thaliana AtGLK1 and AtGLK2 (SEQ ID NOs: 2 and 4); Glycine max G5296 (SEQ ID NO: 6); Oryza sativa G5290 and G5291 (SEQ ID NO: 8 and 10); Physcomitrella patens sequences G5294 and G5295 (SEQ ID NOs: 12 and 14); and Zea mays G5292 and G5293 (SEQ ID NO: 16 and 18).

In addition to the sequences in Tables 1a, 1b, 2a and 2b and the Sequence Listing, the invention encompasses isolated nucleotide sequences that are phylogenetically and structurally similar to sequences listed in the Sequence Listing) and can function in a plant when ectopically expressed by conferring earlier chloroplast development, darker green color as the transgenic plant develops in the absence of light, larger chloroplasts, more extensive chloroplast thylakoid granal development, more carbohydrate, or more chlorophyll.

Since a number of these sequences are phylogenetically and sequentially related to each other and have been shown to enhance plant traits, one skilled in the art would predict that other similar, phylogenetically related sequences falling within the present clades of polypeptides would also perform similar functions when ectopically expressed.

Sequences closely-related to AtGLK1 and AtGLK2 found in various plant species are listed in Tables 1a, 1b, 2a and 2b in descending order of similarity to the Myb-like DNA binding domains of the first-listed sequence in Tables 1a and 2a. These tables include the SEQ ID NO: of the full length protein (Column 1); the species from which each of these phylogenetically-related sequences was derived (Column 2); the Gene Identifier (the name or “GID” of each sequence in Column 3); the percent identity of the polypeptide in Column 1 to the full length AtGLK1 (Table 1a) or AtGLK2 (Table 2a) polypeptide (Column 4); the conserved Myb-like DNA binding domain and the GCT domain amino acid coordinates, respectively, beginning at the n-terminus of each of the protein sequences (Column 5), the SEQ ID NO: of each conserved Myb-like DNA binding domain (Column 6); the conserved Myb-like domain sequences of the respective polypeptides (Column 7); and the percentage identity of the conserved Myb-like domain in Column 7 to the similar Myb-like DNA binding domain of the AtGLK1 or AtGLK2 sequences (Column 8 of Tables 1b and 2b, respectively). Column 8 also includes the ratio of the number of identical residues over the total number of residues compared in the respective Myb-like domains (in parentheses). Columns 9, 10 and 11 respectively list the SEQ ID NO: of each conserved GCT domain, the conserved GCT domain sequences of the respective polypeptides, and the percentage identity of the conserved GCT domain in Column 10 to the similar GCT domain of the AtGLK1 or AtGLK2 sequence. Column 11 also includes the ratio of the number of identical residues over the total number of residues compared in the respective GCT domains (in parentheses).

TABLE 1a
Percentage identities and conserved domains of AtGLK1 and closely related sequences

| | | | Col. 4 | Col. 5 | Col. 6 |
|---|---|---|---|---|---|
| | Col. 2 | Col. 3 | Percent ID | Conserved Myb-like DNA binding | Conserved Myb- |
| Col. 1 | Species from which | Gene ID | of protein | domain and GCT domain amino | like DNA binding |
| SEQ ID NO: | SEQ ID NO: is derived | (GID) | to AtGLK1 | acid coordinates, respectively | domain SEQ ID NO: |
| 2 | Arabidopsis thaliana | AtGLK1 | 100% | 158-206, 370-415 | 19 |
| 6 | Glycine max | G5296 | 46.1 | 175-223, 393-438 | 23 |
| 10 | Oryza sativa | G5291 | 43.6 | 220-268, 487-532 | 27 |
| 12 | Physcomitrella patens | G5294 | 30.4 | 231-279, 469-514 | 29 |
| 14 | Physcomitrella patens | G5295 | 29.3 | 227-275, 463-508 | 31 |
| 4 | Arabidopsis thaliana | AtGLK2 | 46.9 | 152-200, 339-384 | 21 |
| 16 | Zea mays | G5292 | 43.0 | 189-237, 406-451 | 33 |
| 8 | Oryza sativa | G5290 | 44.3 | 185-233, 407-452 | 25 |
| 18 | Zea mays | G5293 | 41.9 | 198-246, 427-472 | 35 |

TABLE 1b

Percentage identities and conserved domains of AtGLK1 and
closely related sequences

| | | Col. 8 | | | |
|---|---|---|---|---|---|
| | | Percent ID of | | | |
| | | Myb-like DNA | | | |
| Col. 1 | | binding domain | Col. 9 | | Col. 11 |
| SEQ | Col. 7 | to AtGLK1 | Conserved | Col. 10 | Percent ID of GCT |
| ID | Conserved Myb-like | Myb-like DNA | GCT domain | Conserved | domain to AtGLK1 |
| NO: | DNA binding domain | binding domain | SEQ ID NO: | GCT domain | GCT domain |
| 2 | WTPELHRRFVEA | 100% (48/48) | 20 | SKESVDAAIG | 100% (46/46) |
| | VEQLGVDKAVPS | | | DVLTRPWLP | |
| | RILELMGVHCLT | | | LPLGLNPPAV | |
| | RHNVASHLQKYR | | | DGVMTELHR | |
| | S | | | HGVSEVPP | |
| 6 | WTPELHRRFVQAV | 89% (44/49) | 24 | SKESIDAAISD | 69% (32/46) |
| | EQLGVDKAVPSRIL | | | VLSKPWLPLP | |
| | EIMGIDCLTRHNIAS | | | LGLKAPALD | |
| | HLQKYRS | | | GVMGELQRQ | |
| | | | | GIPKIPP | |
| 10 | WTPELHRRFVQAV | 89% (44/49) | 28 | SKESIDAAIG | 71% (33/46) |
| | EQLGIDKAVPSRILE | | | DVLVKPWLP | |
| | LMGIECLTRHNIAS | | | LPLGLKPPSL | |
| | HLQKYRS | | | DSVMSELHK | |
| | | | | QGIPKVPP | |
| 12 | WTPELHRRFVHAV | 89 (44/49) | 30 | SKEVLDAAIG | 58% (27/46) |
| | EQLGVEKAYPSRIL | | | EALANPWTP | |
| | ELMGVQCLTRHNI | | | PPLGLKPPSM | |
| | ASHLQKYRS | | | EGVIAELQRQ | |
| | | | | GINTVPP | |
| 14 | WTPELHRRFVHAV | 89% (44/49) | 32 | SKEVLDAAIG | 58% (27/46) |
| | EQLGVEKAFPSRIL | | | EALANPQTP | |
| | ELMGVQCLTRHNI | | | PPLGLKPPSM | |
| | ASHLQKYRS | | | EGVIAELQRQ | |
| | | | | GINTVPP | |
| 4 | WTPELHRKFVQAV | 87% (43/49) | 22 | SNESIDAAIG | 78% (36/46) |
| | EQLGVDKAVPSRIL | | | DVISKPWLPL | |
| | EIMNVKSLTRHNV | | | PLGLKPPSVD | |
| | ASHLQKYRS | | | GVMTELQRQ | |
| | | | | GVSNVPP | |
| 16 | WTPELHRRFVQAV | 87% (43/49) | 34 | SKESIDAAIG | 71% (33/46) |
| | EQLGIDKAVPSRILE | | | DVLVKPWLP | |
| | IMGTDCLTRHNIAS | | | LPLGLKPPSL | |
| | HLQKYRS | | | DSVMSELHK | |
| | | | | QGVPKIPP | |
| 8 | WTPELHRRFVQAV | 85% (42/49) | 26 | SSESIDAAIGD | 73% (34/46) |
| | EQLGIDKAVPSRILE | | | VLSKPWLPLP | |
| | IMGIDSLTRHNIASH | | | LGLKPPSVDS | |
| | LQKYRS | | | VMGELQRQG | |
| | | | | VANVPP | |
| 18 | WTPELHRRFVQAV | 83% (41/49) | 36 | SSESIDAAIGD | 73% (34/46) |
| | EELGIDKAVPSRILE | | | VLTKPWLPLP | |
| | IMGIDSLTRHNIASH | | | LGLKPPSVDS | |
| | LQKYRS | | | VMGELQRQG | |
| | | | | VANVPQ | |

Similar to Tables 1a and 1b, Tables 2a and 2b compare AtGLK2 to full-length proteins and conserved domains of closely related sequences

TABLE 2a
Percentage identities and conserved domains of AtGLK2 and closely related sequences

| | | | Col. 4 | Col. 5 | Col. 6 |
|---|---|---|---|---|---|
| | Col. 2 | Col. 3 | Percent ID | Conserved Myb-like DNA binding | Conserved Myb- |
| Col. 1 | Species from which | Gene ID | of protein | domain and GCT domain amino | like DNA binding |
| SEQ ID NO: | SEQ ID NO: is derived | (GID) | to AtGLK2 | acid coordinates, respectively | domain SEQ ID NO: |
| 4 | Arabidopsis thaliana | AtGLK2 | 100% | 152-200, 339-384 | 21 |
| 2 | Arabidopsis thaliana | AtGLK1 | 46.9 | 158-206, 370-415 | 19 |
| 6 | Glycine max | G5296 | 44.4 | 175-223, 393-438 | 23 |
| 8 | Oryza sativa | G5290 | 44.3 | 185-233, 407-452 | 25 |
| 16 | Zea mays | G5292 | 43.6 | 189-237, 406-451 | 33 |
| 18 | Zea mays | G5293 | 42.2 | 198-246, 427-472 | 35 |
| 10 | Oryza sativa | G5291 | 41.7 | 220-268, 487-532 | 27 |
| 12 | Physcomitrella patens | G5294 | 32.4 | 231-279, 469-514 | 29 |
| 14 | Physcomitrella patens | G5295 | 33.2 | 227-275, 463-508 | 31 |

TABLE 2b

Percentage identities and conserved domains of AtGLK2 and
closely related sequences

| | | Col. 8 | | | |
|---|---|---|---|---|---|
| | | Percent ID of | | | |
| | | Myb-like DNA | | | |
| Col. 1 | | binding domain | Col. 9 | | Col. 11 |
| SEQ | Col. 7 | to AtGLK2 | Conserved | Col. 10 | Percent ID of GCT |
| ID | Conserved Myb-like | Myb-like DNA | GCT domain | Conserved | domain to AtGLK2 |
| NO: | DNA binding domain | binding domain | SEQ ID NO: | GCT domain | GCT domain |
| 4 | WTPELHRKFVQAVEQLGV | 100% (49/49) | 22 | SNESIDAAIGDVI | 100% (46/46) |
| | DKAVPSRILEIMNVKSLT | | | SKPWLPLPLGLK | |
| | RHNVASHLQKYRS | | | PPSVDGVMTEL | |
| | | | | QRQGVSNVPP | |
| 2 | WTPELHRRFVEAVEQLG | 87% (43/49) | 20 | SKESVDAAIGDV | 78% (36/46) |
| | VDKAVPSRILELMGVHC | | | LTRPWLPLPLGL | |
| | LTRHNVASHLQKYRS | | | NPPAVDGVMTE | |
| | | | | LHRHGVSEVPP | |
| 6 | WTPELHRRFVQAVEQLGV | 87% (43/49) | 24 | SKESIDAAISDVL | 76% (35/46) |
| | DKAVPSRILEIMGIDCLT | | | SKPWLPLPLGLK | |
| | RHNIASHLQKYRS | | | APALDGVMGEL | |
| | | | | QRQGIPKIPP | |
| 8 | WTPELHRRFVQAVEQLGID | 87% (43/49) | 26 | SSESIDAAIGDVL | 89% (41/46) |
| | KAVPSRILEIMGIDSLTRH | | | SKPWLPLPLGLK | |
| | NIASHLQKYRS | | | PPSVDSVMGEL | |
| | | | | QRQGVANVPP | |
| 16 | WTPELHRRFVQAVEQLGID | 85% (42/49) | 36 | SSESIDAAIGDVL | 84% (39/46) |
| | KAVPSRILEIMGIDCLTRH | | | LVKPWLPLPLGL | |
| | NIASHLQKYRS | | | KPPSLDSVMSEL | |
| | | | | HKQGVPKIPP | |
| 18 | WTPELHRRFVQAVEELGID | 85% (42/49) | 36 | SSESIDAAIGDVL | 84% (39/46) |
| | KAVPSRILEIMGIDSLTRH | | | TKPWLPLPLGLK | |
| | NIASHLQKYRS | | | PPSVDSVMGEL | |
| | | | | QRQGVANVPQ | |
| 10 | WTPELHRRFVQAVEQLGID | 83% (41/49) | 28 | SKESIDAAIGDV | 76% (35/46) |
| | KAVPSRILELMGIECLTRH | | | LVKPWLPLPLGL | |
| | NIASHLQKYRS | | | KPPSLDSVMSEL | |
| | | | | HKQGIPKVPP | |
| 12 | WTPELHRRFVHAVEQLGVE | 81% (40/49) | 30 | SKEVLDAAIGEA | 63% (29/46) |
| | KAYPSRILELMGVQCLTRH | | | LANPWTPPPLGL | |
| | NIASHLQKYRS | | | KPPSMEGVIAEL | |
| | | | | QRQGINTVPP | |
| 14 | WTPELHRRFVHAVEQLGVE | 81% (40/49) | 32 | SKEVLDAAIGEA | 63% (29/46) |
| | KAFPSRILELMGVQCLTRH | | | LANPWTPPPLGL | |
| | NIASHLQKYRS | | | KPPSMEGVIAEL | |
| | | | | QRQGINTVPP | |

### Sequence Variations

It will readily be appreciated by those of skill in the art, that the invention includes any of a variety of polynucleotide sequences provided in the Sequence Listing or capable of encoding polypeptides that function similarly to those provided in the Sequence Listing. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (that is, peptides having some degree of equivalent or similar biological activity) but differ in sequence from the sequence shown in the sequence listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide. For example, substitutions, deletions and insertions introduced into the sequences provided in the Sequence Listing are also envisioned. Such sequence modifications can be engineered into a sequence by site-directed mutagenesis (for example, Olson et al., Smith et al., Zhao et al., and other articles in Wu (ed.) Meth. Enzymol. (1993) vol. 217, Academic Press) or the other methods known in the art or noted herein. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as conservative substitutions.

TABLE 3
Possible conservative amino acid substitutions

| Amino Acid Residue | Conservative substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

The polypeptides provided in the Sequence Listing have a novel activity, such as, for example, regulatory activity. Although all conservative amino acid substitutions (for example, one basic amino acid substituted for another basic amino acid) in a polypeptide will not necessarily result in the polypeptide retaining its activity, it is expected that many of these conservative mutations would result in the polypeptide retaining its activity. Most mutations, conservative or non-conservative, made to a protein but outside of a conserved domain required for function and protein activity will not affect the activity of the protein to any great extent.

### EXAMPLES

### Example I

### Cloning Information

A number of constructs were used or may be used to modulate the activity of sequences of the invention. Analysis of plants is typically performed on a set of independent transgenic lines (also known as “events”) which are stably transformed with a particular construct (for example, this might include plant lines that constitutively overexpress AtGLK1, AtGLK2 or an ortholog or another clade polypeptide). Generally, a full-length wild-type version of a gene or its cDNA is directly fused to a promoter that drives its expression in transgenic plants. Such a promoter can be the native promoter of that gene, or a promoter that drives constitutive expression such as the CaMV 35S promoter. Alternatively, a promoter that drives tissue-enhanced or conditional expression can be used in similar studies. A direct fusion approach has the advantage of allowing for simple genetic analysis if a given promoter-polynucleotide line is to be crossed into different genetic backgrounds at a later date.

As an alternative to plant transformation with a direct fusion construct, transgenic plant lines may be generated that express the gene of interest by means of a two component expression system comprising two different transgenes that are integrated into the plant DNA: the first of these is a transcriptional activator component (the “driver”) such as a Promoter::LexA-GAL4-TA (where the promoter drives expression in the pattern of interest) and the second is a responder component that is targeted by the transcriptional activator, such as an opLexA::transcription factor expression cassette. The two components may be brought together in the same plant by crossing or super-transformation.

As an example, the first component vector, the “driver” vector or construct (e.g., P6506, P5287, P5284, or P5303, SEQ ID NOs: 42, 39, 40, or 41, respectively) contains a transgene carrying a Promoter::LexA-GAL4-transactivation domain (TA) along with a resistance selectable marker (e.g., a kanamycin resistance marker). Having established a driver line containing this Promoter::LexA-GAL4-transactivation domain component, the transcription factors of the invention can be expressed by super-transforming or crossing in a second construct carrying e.g., a sulphonamide resistance selectable marker and the transcription factor polynucleotide of interest cloned behind a LexA operator site (opLexA::TF). For example, the two constructs P6506 (35S::LexA-GAL4TA; SEQ ID NO: 42) and P7446 (opLexA::AtGLK1; SEQ ID NO: 37) together constitute a two-component system for expression of AtGLK1 from the 35S promoter. A kanamycin resistant transgenic line containing P6506 is established, and this is then supertransformed with the P7446 construct containing a genomic clone of AtGLK1 and a sulfonamide resistance marker. For each transcription factor that is overexpressed with a two component system, the second construct carries a second (e.g., sulfonamide) selectable marker.

Promoters used in nucleic acid constructs that may be used to regulate ectopic expression of AtGLK1-related sequences should be selected from a set of promoters that function in the plant species of interest.

### Example II

### Tomato Lines, Fruit Staging and Harvesting

Transgenic tomato (Solanum lycopersicum) lines expressing transcription factors AtGLK1 (At2g20570) or AtGLK2 (At5g44190) regulated by the 35S, LTP, Phytoene desaturase (PD), or RbcS, promoters were grown in greenhouse and field trials in Davis, Calif. between 2004 and 2006. The identity of the transgenic constructs in each line was confirmed by PCR using primers for the selectable marker, each promoter and each transcription factor. Fruit were tagged 3-4 days after anthesis when they were 0.5 cm diameter, to obtain material from the same developmental stage. Mature green and red ripe fruit were harvested 32 and 46 days after tagging respectively.

To determine the role of light for the development of green color, 4 days after anthesis (0.5 cm diameter) fruit were placed in paper envelopes that blocked 80% of the light for two weeks and then the bags then were replaced with bags with three layers (white (external), black and red) that blocked 100% of the light until the fruit were harvested. Fruit were compared to fruit tagged at the same time but not contained in light-blocking bags.

### Example III

### Biochemical and Morphological Analyses

Chlorophyll content. Chlorophyll was measured in fully expanded apical leaves and in mature green and red fruit. Tissue from the outer fruit pericarp and epidermis (0.25 g) was crushed in liquid nitrogen. One ml of 90% acetone was added to the frozen powder and the mixture shaken at room temperature in the dark overnight. After centrifugation for 10 minutes to remove the colorless cellular debris, the chlorophyll contents of a 1:5 (v:v) dilution (using 90% acetone) of the supernatant was measured using the absorbance at 645 nm for chlorophyll b and 663 nm for chlorophyll a and the amount of Chl a or Chl b was calculated according to Arnon (1949). Total chlorophyll was calculated as Chl a+Chl b. Results were expressed as μg chlorophyll per gram fresh weight (g fw) tissue extracted.

Lycopene measurement. Lycopene was measured in red ripe fruit. Frozen tissue from the outer fruit pericarp (0.25 g) was crushed in liquid nitrogen and added to 1.5 ml of 4:3 ethanol: hexane (v:v) in foil covered tubes. The tubes were shaken for 4 h at room temperature until the pigments were totally extracted. After centrifugation to remove the cellular debris, the supernatant was diluted 1:5 (v:v) with the ethanol:hexane mixture. The absorbance at 510 nm was measured and the results were expressed as μg g⁻¹ fw using an extinction coefficient of 3450 E^{1%} 1 cm (Periago et al., 2007).

Starch measurements and staining. Two grams of fruit outer pericarp were ground in 10 ml ethanol. The samples were centrifuged and the pellet was re-extracted two more times with 10 ml ethanol. After centrifugation the pellet was dried at 50° C. and resuspended in 5 ml of Na acetate buffer, pH 5.0, 50 mM. One hundred microliters of a solution containing 10 units of amylase and 3 units of amyloglucosidase were added and incubated at 30° C. with stirring overnight. The samples were centrifuged and adjusted to 6 ml with water. The content of reducing sugars was determined spectrophotometrically at 520 nm using a modification of the Somogyi-Nelson method (Southgate, 1976).

To stain visibly for starch, fruit slices from control, AtGLK1- and AtGLK2-expressing lines at 3 developmental stages (immature green with diameters of 1 cm, about seven days post anthesis, 2.5 cm, about 14 days post anthesis, or mature green) were cut with a razor blade and incubated for 5 min in a solution containing 1% I₂ and 2% KI. After 5 min samples were taken rinsed with distilled water and photographed.

Soluble solids and sugar measurements. Soluble solids were measured using fresh fruit juice from freshly harvested red ripe fruit. A handheld digital refractometer (PR100, Atago Co., Ltd., Tokyo) was used. For simple sugar analysis 5 to 7 g of fruit were extracted with 20 ml ethanol. The samples were centrifuged and re-extracted with 10 ml of ethanol. The supernatants were pooled and taken to 45 ml. Two hundred microliters of sample were dried and resuspended in 1 ml. Forty microliters of sample was then taken to 10 ml and 200 microliters were injected in the HPLC for sugar analysis. Sugar profiles were analyzed using a DX-500 HPLC system (Dionex) equipped with an ED-40 pulsed amperometric detector (Dionex). Sugars were separated on a Carbopac™ PA1 column, using linear sodium acetate gradient at a flow rate of 0.6 ml/min.

Transmission electron microscopy. Pericarp fragments were excised from fruit at the immature green, mature green and red ripe stages and from fully expanded leaves. Fragments were fixed in Karnovsky's fixative using vacuum-microwave combination as described by Russin and Trivett (Russin and Trivett, 2001) and washed in 0.1M sodium phosphate buffer, pH 7.2, microwaved under vacuum at 450 W for 40 seconds, post-fixed for 2 hours in 1% osmium tetroxide buffered in 0.1M sodium phosphate buffer and microwaved a second time at 450 W for 40 seconds. After incubation in 0.1% tannic acid in water for 30 minutes on ice and in 2% aqueous uranyl acetate for 1 hour, samples were dehydrated in acetone and embedded in Epon/Araldite resin. Ultrathin sections were examined with a Philips CM120 Biotwin Lens transmission electron microscope (FEI Company, Hillsboro, Oreg.).

### Example IV

### Effects of Expression of AtGLK1 or AtGLK2 on Fruit Color and Chlorophyll Content

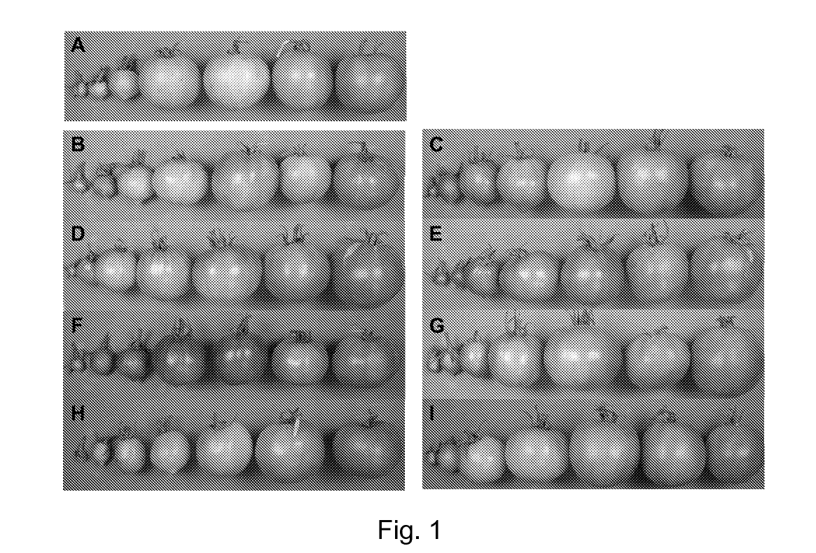

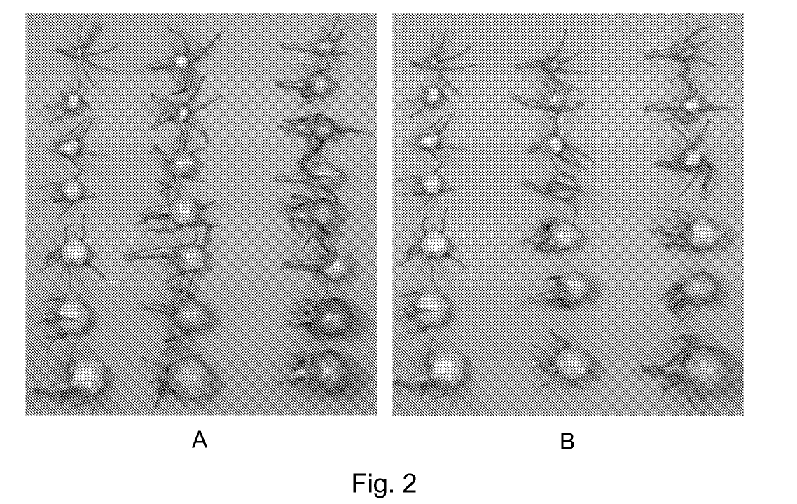

Increased green color of fruit before ripening. During two years of field trials for surveying the phenotypes in a large population of transgenic tomato lines expressing Arabidopsis transcription factors under the control of four promoters, two transcription factors, when expressed with each of the promoters, were notable for conferring a particularly dark green fruit phenotype, as compared to control plants (FIG. 1). The intensity of the green hue of the fruit varied depending on the promoter controlling expression of the transcription factor. Expression of AtGLK1 with the rubisco small subunit (RbcS) promoter produced the most intensely green AtGLK1-expressing fruit and expression with the lipid transfer protein (LTP) promoter produced the most intensely green AtGLK2-expressing fruit. Expression with the phytoene desaturase (PD) promoter caused the least dark green fruit with either transcription factor but these fruit were still noticeably greener than control fruit. In very young fruit, expression of either AtGLK1 or AtGLK2 with the RbcS promoter gave the most intensely green very young fruit (FIG. 2). Very young fruit expressing AtGLK1 or AtGLK2 with either the LTP or the RbcS promoter were more intensely green than control fruit of the same age.

Sequencing of PCR products from the lines with dark green fruit identified AtGLK1 (At2g20570) and AtGLK2 (At5g44190) as the Arabidopsis transcription factors expressed in these lines and confirmed the identity of the promoters in the lines.

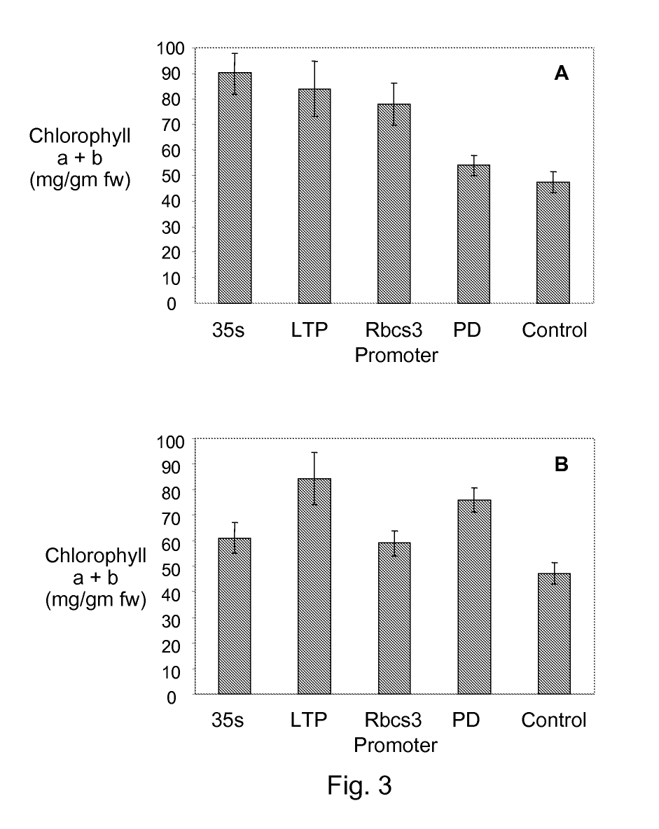

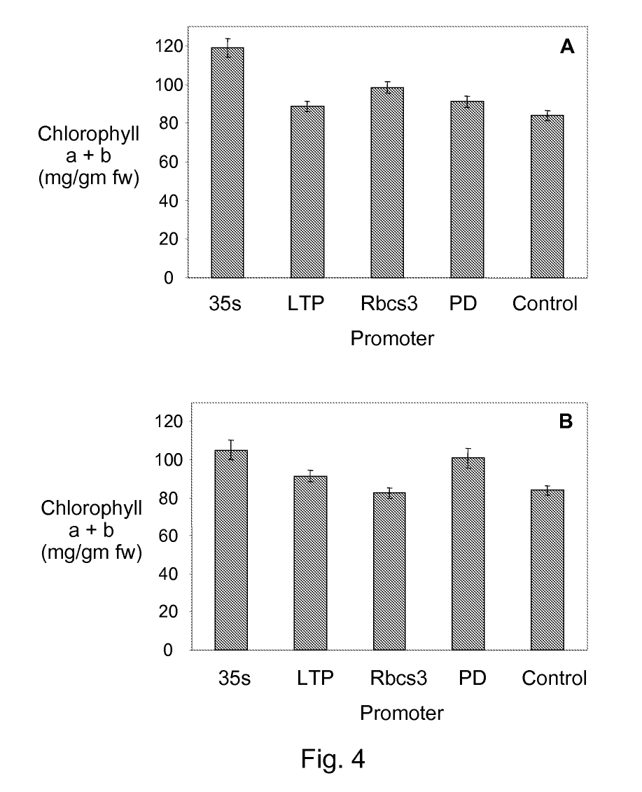

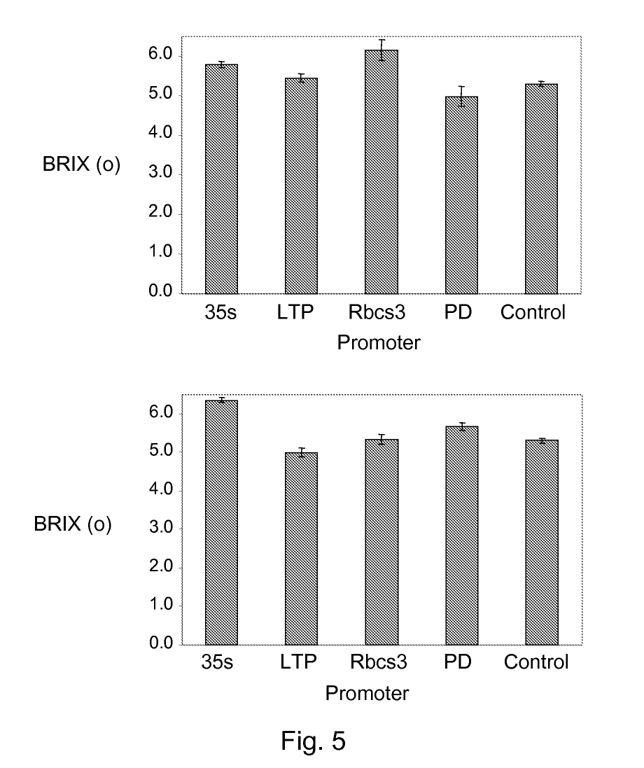

The chlorophyll contents of the leaves and the fruit pericarp were examined. All of the transgenic lines expressing AtGLK1 or AtGLK2 had significantly higher amounts of total chlorophyll (chlorophyll a+b) in mature green fruit than the control lines (FIGS. 3A and 3B). The amount of chlorophyll varied depending on the promoter expressing AtGLK1 or AtGLK2. Notably, fruit from plants with AtGLK1 expressed from the 35S promoter had about 100% more chlorophyll than control fruit. Fruit from plants carrying AtGLK2 expressed from the same 35S promoter construct had about 30% more chlorophyll a than control fruit. Chlorophyll content in the leaves was also higher in the transgenic lines expressing AtGLK1 or AtGLK2 compared to the control (FIGS. 4A and 4B) although the increases were substantially less than those observed in fruits. The chlorophyll a/b ratios were not different from that found in control fruit suggesting that no preferential modification of either of the photosystems occurred (Table 4). Analysis of the lycopene in the red ripened fruit in the transgenic lines showed little difference in the amount of lycopene between the lines and compared to control fruit (FIGS. 5A, 5B), although the lines expressing AtGLK1 had in some cases slightly less lycopene than control fruit.

Table 4 provides chlorophyll a and chlorophyll b contents and chlorophyll a:b ratio determined in leaves and immature and mature green fruit from plants expressing AtGLK1 or AtGLK2. Chlorophyll is expressed as mg/g fresh weight.

TABLE 4
Chlorophyll a and chlorophyll b contents and chlorophyll a:b ratio
determined in leaves and immature and mature green fruit

| | | Immature Green Fruit | | | Mature Green Fruit | | |
|---|---|---|---|---|---|---|---|
| | Promoter | Chl a | Chl b | Ratio | Chl a | Chl b | Ratio |
| Control | | 29.66 ± 2.37 | 11.74 ± 0.86 | 2.54 ± 0.03 | 21.96 ± 3.05 | 25.30 ± 5.27 | 1.27 ± 0.10 |
| AtGLK1 | 35S | 29.24 ± 0.57 | 12.17 ± 0.49 | 2.36 ± 0.07 | 42.79 ± 5.10 | 47.66 ± 10.84 | 1.49 ± 0.03 |
| | LTP | 29.48 ± 6.13 | 12.25 ± 2.28 | 2.40 ± 0.06 | 41.08 ± 8.44 | 42.99 ± 13.25 | 1.20 ± 0.09 |
| | RBCs3 | 32.65 ± 11.94 | 12.73 ± 2.83 | 2.48 ± 0.27 | 38.88 ± 5.92 | 38.96 ± 10.52 | 1.25 ± 0.11 |
| | PD | 24.13 ± 4.30 | 10.46 ± 1.90 | 2.32 ± 0.02 | 34.07 ± 5.89 | 19.87 ± 1.96 | 1.70 ± 0.09 |
| AtGLK2 | 35S | 72.98 ± 32.14 | 68.29 ± 42.78 | 1.81 ± 0.28 | 27.85 ± 2.90 | 33.20 ± 9.15 | 1.35 ± 0.08 |
| | LTP | 36.59 ± 2.01 | 16.37 ± 1.29 | 2.27 ± 0.03 | 39.81 ± 6.61 | 44.83 ± 13.55 | 1.27 ± 0.04 |
| | RBCs3 | 34.24 ± 4.96 | 21.12 ± 7.76 | 2.18 ± 0.07 | 32.22 ± 4.54 | 26.83 ± 5.42 | 1.41 ± 0.06 |
| | PD | 23.13 ± 2.81 | 10.97 ± 1.09 | 2.19 ± 0.12 | 37.76 ± 4.22 | 38.28 ± 5.17 | 1.06 ± 0.05 |

| | | Leaves | | |
|---|---|---|---|---|
| | Promoter | Chl a | Chl b | Ratio |
| Control | | 65.39 ± 3.49 | 18.55 ± 1.73 | 3.52 ± 2.02 |
| AtGLK1 | 35S | 78.31 ± 6.21 | 40.50 ± 3.24 | 1.93 ± 1.92 |
| | LTP | 68.07 ± 3.09 | 20.34 ± 2.24 | 3.35 ± 1.38 |
| | RBCs3 | 77.66 ± 3.93 | 20.72 ± 1.84 | 3.75 ± 2.14 |
| | PD | 72.17 ± 4.20 | 18.88 ± 1.89 | 3.82 ± 2.23 |
| AtGLK2 | 35S | 79.24 ± 6.72 | 25.83 ± 3.47 | 3.07 ± 1.94 |
| | LTP % | 70.79 ± 3.92 | 20.60 ± 2.35 | 3.44 ± 1.66 |
| | RBCs3 | 65.19 ± 3.27 | 17.31 ± 2.28 | 3.77 ± 1.43 |
| | PD | 73.04 ± 5.54 | 27.78 ± 5.24 | 2.63 ± 2.02 |

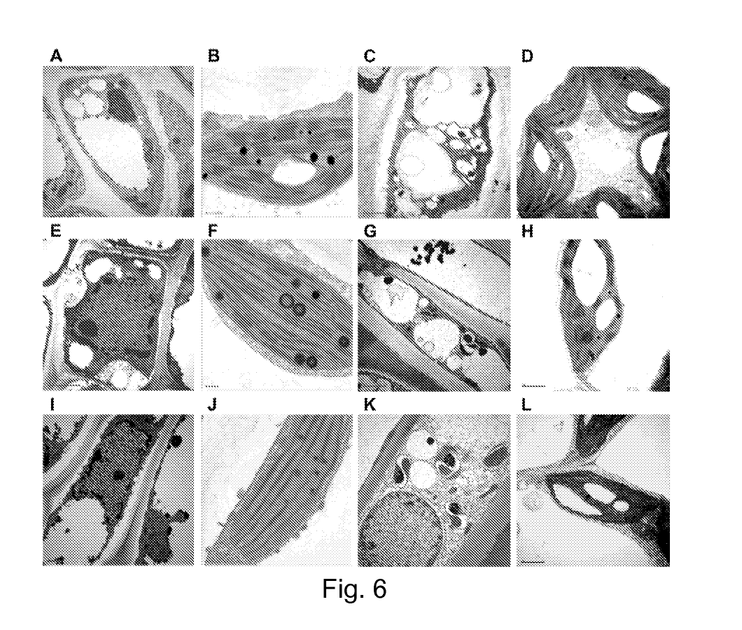

Expression of AtGLK1 or AtGLK2 alters the chloroplast structure in green fruit. Since the chlorophyll content of the green fruit expressing AtGLK1 or AtGLK2 was so markedly increased relative to control fruit, microscopic analysis of the chloroplast structure was used to assess further the consequences of AtGLK1 or AtGLK2 expression. To simplify the analysis, only fruit from lines expressing the transcription factors by the 35S promoter (35S::AtGLK1 or 35S::AtGLK2) and grown in the greenhouse were examined. Light microscopy of fruit pericarp cells suggested that chloroplasts from 35S::AtGLK1 expressing fruit were substantially denser, and from 35S::AtGLK2 expressing fruit were somewhat less but still perceptibly denser, than chloroplasts from mature green control fruit (data not shown). Transmission electron microscopy of chloroplasts from mature green fruit confirmed this observation and showed that the chloroplasts from green fruit expressing 35S::AtGLK1 were larger; more rounded and, most noticeably, contained thylakoid membranes with large granal stacks (FIG. 6B). Chloroplasts from mature green fruit expressing 35S::AtGLK2 were also larger than those from control mature green fruit but the granal stacking was not as pronounced as in the fruit expressing 35S::AtGLK1 (FIG. 6F). Chloroplasts from either the 35S::AtGLK1 or 35S::AtGLK2 expressing mature green fruit had a higher frequency of starch bodies and plastoglobule granules than chloroplasts from control fruit. Mature green fruit pericarp from 35S::AtGLK1 or 35S::AtGLK2 expressing lines contained approximately twice as many chloroplasts as cells from similar control tissues. Immature green fruit expressing 35S::AtGLK1 contained more identifiable chloroplasts than immature green control or 35S::AtGLK2-expressing fruit. No differences in chloroplast or chromoplast structure were observed in leaves or in red fruit between the 35S::AtGLK1 or 35S::AtGLK2 and control lines.

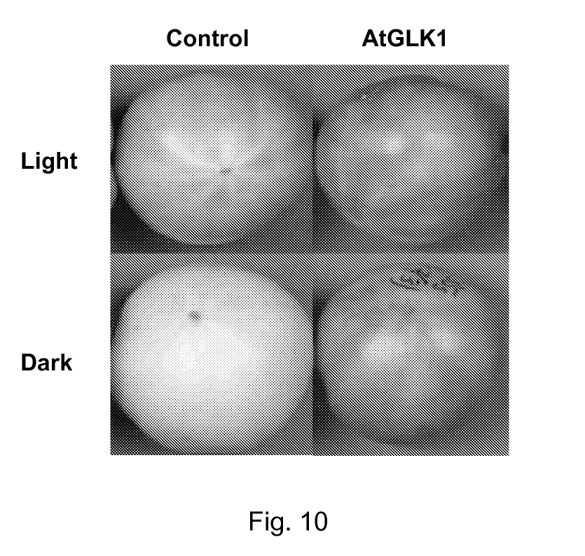

Expression of AtGLK1 causes fruit to remain green in the absence of light. Enclosing developing wild-type fruit in light-blocking paper bags results in fruit with little chlorophyll (FIG. 10). However, fruit expressing 35S::AtGLK1 were almost as green as fruit that had developed in the sunlight when subjected to such treatments (FIG. 10), suggesting that AtGLK1 and homologous proteins with similar activity may function as a photomorphogenic signal, or regulate the plant responses to such signals.

### Example V

### Expression of AtGLK1 or AtGLK2 Increases Starch and Sugar Accumulation in Fruit

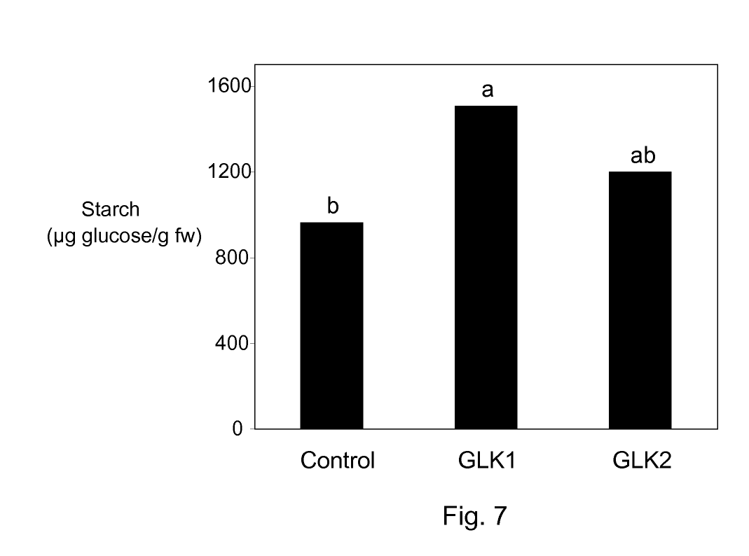

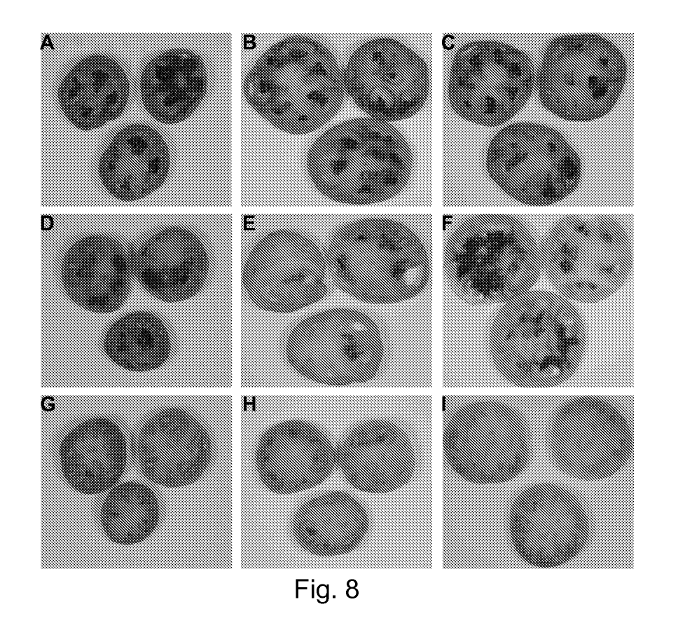

The amount of starch was measured in pericarp from immature, mature green fruit and leaves from plants expressing 35S::AtGLK1 or 35S::AtGLK2. Immature green fruit from both transgenic lines contained more starch than immature green fruit from control lines, although the increase was only statistically significant only for the 35S::AtGLK1 fruit (FIG. 7). Iodide staining of slices of developing green fruit demonstrated, however, that both 35S::AtGLK1 and 35S::AtGLK2 expressing green fruit contained much more starch in the locular region than did control green fruit (FIG. 8). Similar results were obtained for green fruit expressing either AtGLK1 or AtGLK2 with the RbcS promoter.

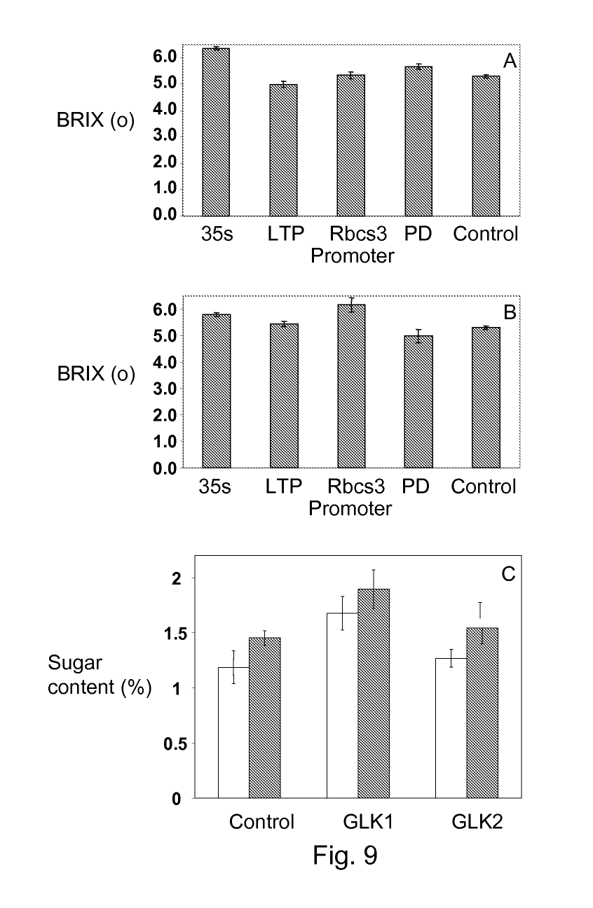

To measure whether the expression of AtGLK1 or AtGLK2 influenced the accumulation of sugars in the ripe fruit, the BRIX in the red ripe fruit juice was measured (FIG. 9A). Expression of 35S::AtGLK1 resulted in a 21% increase in BRIX in red fruit compared to control red fruit. 35S::AtGLK1 expressing red fruit had a 40% increase in sucrose and glucose compared to control red fruit (FIG. 9C). Expression of 35S::AtGLK2 resulted in a smaller increase in sugars and BRIX (FIG. 9B).

### Example VI

### Transgenic Plants with Elevated Carbohydrate or Chlorophyll Levels in Various Plant Organs

Transgenic plants, for example, soybean, overexpressing AtGLK1 (SEQ ID NO: 2), or AtGLK2 (SEQ ID NO: 4), or orthologs of these sequences, e.g., Glycine max G5296 (SEQ ID NO: 6), Oryza sativa G5290 and G5291 (SEQ ID NO: 8 and 10), Physcomitrella patens sequences G5294 and G5295 (SEQ ID NOs: 12 and 14), or Zea mays G5292 and G5293 (SEQ ID NO: 16 and 18) or other sequences from other plant species determined to be orthologous to AtGLK1 or AtGLK2, may be produced according to methods described herein. These transgenic plants may have elevated carbohydrate levels in organs such as leaves or seeds with respect to a control plant (e.g., a wild type plant, a plant transformed with an empty vector, or a plant of the same species that does not have the recombinant polynucleotide that encodes the GLK-related polypeptide). The elevated carbohydrate levels may include increased starch and increased levels of sugars such as sucrose and fructose.

Starch levels may be assessed by iodide staining, using methods known in the art or provided above.

Although the methodologies described herein are provided as examples, this description is not to be limited by those provided therein. Those skilled in the art will understand that alternative methods exist that may be used. For example, the method to measure soluble sugars may depend on the carbohydrate being measured and depth of analysis (e.g., total carbohydrate content or individual carbohydrate content).

One method of measuring soluble sugars is through the use of refractometry. A refractometer is an optical instrument used to measure the concentration or refractive index of liquids. The tomato sample is filtered, and a drop of the filtrate is used to measure the refractive index. The extent of refraction is dependent on the amount of sugar.

Soluble sugars may also be separated from sugar polymers by extracting plant tissues such as leaves, roots, or stems with hot 70% ethanol. Carbohydrate content can then be estimated using a variety of techniques such as high performance liquid chromatography (HPLC; using either electrochemical or refractive index detectors) or gas chromatography (GC; with derivatization to make the carbohydrates volatile). In certain cases the carbohydrate content can be analyzed enzymatically or colorimetrically.

Chlorophyll may be estimated using in methanolic extracts using the method of Porra et al. (1989). or with, for example, a Minolta SPAD-502 (Konica Minolta Sensing Americas, Inc., Ramsey, N.J.). Chlorophyll content and amount can also be determined with HPLC. Pigments are extracted from leave tissue by homogenizing leaves in acetone:ethyl acetate (3:2). Water is added, the mixture centrifuged, and the upper phase removed for HPLC analysis. Samples can be analyzed using a Zorbax (Agilent Technologies, Palo Alto, Calif.) C18 (non-endcapped) column (250×4.6) with a gradient of acetonitrile:water (85:15) to acetonitrile:methanol (85:15) in 12.5 minutes. After holding at these conditions for two minutes, solvent conditions are changed to methanol:ethyl:acetate (68:32) in two minutes. Chlorophylls are quantified using peak areas and response factors calculated using β-carotene as the standard.

Transgenic plants that may be transformed with AtGLK1 (SEQ ID NO: 2), or AtGLK2 (SEQ ID NO: 4), or orthologs of those genes and express the useful traits described herein include, but are not limited to, dicots, including soybean, potato, cotton, rape, oilseed rape (including canola), sunflower, alfalfa, fruits and vegetables such as banana, blackberry, blueberry, strawberry, raspberry, cantaloupe, carrot, cauliflower, coffee, cucumber, eggplant, grapes, honeydew, lettuce, mango, melon, onion, papaya, peas, peppers, pineapple, pumpkin, spinach, squash, tobacco, tomato, watermelon, rosaceous fruits (such as apple, peach, pear, cherry and plum) vegetable brassicas (such as broccoli, cabbage, cauliflower, Brussels sprouts, kohlrabi, currant, avocado, citrus fruits such as oranges, lemons, grapefruit and tangerines, artichoke, cherries, nuts such as the walnut and peanut, endive, leek, root, such as arrowroot, beet, cassava, turnip, radish, yam, sweet potato, beans, woody species such pine, poplar and eucalyptus, or mint or other labiates, and monocots, including but not limited to wheat, corn, sweet corn, rice, sugarcane, turfgrass; barley, rye, millet, sorghum, Miscanthus, and switchgrass.

### REFERENCES CITED

Altschul (1990) J. Mol. Biol. 215: 403-410.
Altschul (1993) J. Mol. Evol. 36: 290-300.
Altschul et al. (1997) Nucleic Acids Res. 25: 3389-3402.
Arnon (1949) Plant Physiol. 24: 1-15.
Ausubel et al. (1997) Short Protocols in Molecular Biology, John Wiley & Sons, New York, N.Y., unit 7.7.
Bairoch et al. (1997) Nucleic Acids Res. 25: 217-221.
Bhattacharjee et al. (2001) Proc. Natl. Acad. Sci. USA 98: 13790-13795.
Blanke and Lenz (1989) Plant Cell Environ. 12: 31-46.
Boss and Thomas (2002) Nature 416: 847-850.
Bruce et al. (2000) Plant Cell 12: 65-79.
Borevitz et al. (2000) Plant Cell 12: 2383-2393.
Carrara et al. (2001) Photosynthetica 39: 75-78.
Coupland (1995) Nature 377: 482-483.
Cribb et al. (2001) Genetics 159: 787-797.
Dayhoff et al. (1978) “A model of evolutionary change in proteins,” in: Atlas of Protein Sequence and Structure, Vol. 5, Suppl. 3 (ed. M. O. Dayhoff), pp. 345-352. Natl. Biomed. Res. Found., Washington, D.C.
Doolittle, ed. (1996) Methods in Enzymology, vol. 266: “Computer Methods for Macromolecular Sequence Analysis” Academic Press, Inc., San Diego, Calif., USA.
Eddy (1996) Curr. Opin. Str. Biol. 6: 361-365.
Edwards and Huber (1979) C4 metabolism in isolated cells and protoplasts. In MGaE Latzko, ed, Encyclopedia of Plant Physiology. Springer-Verlag, New York, pp 102-112.
Eisen (1998) Genome Res. 8: 163-167.
Feng and Doolittle (1987) J. Mol. Evol. 25: 351-360.
Fitter et al. (2002) Plant J. 31: 713-727.
Fowler and Thomashow (2002) Plant Cell 14: 1675-1690.
Fu et al. (2001) Plant Cell 13: 1791-1802.
Gillaspy et al. (1993) Plant Cell 5: 1439-1451.
Gilmour et al. (1998) Plant J. 16: 433-442.
Giovannoni (2007) Curr. Opin. Plant Biol. 10: 283-289.
Goodrich et al. (1993) Cell 75: 519-530.
Hall et al. (1998) Plant Cell 10: 925-936.
Haymes et al. “Nucleic Acid Hybridization: A Practical Approach”, IRL Press, Washington, D.C. (1985).
He et al. (2000) Transgenic Res. 9: 223-227.
Hein (1990) Methods Enzymol. 183: 626-645.
Henikoff and Henikoff (1991) Nucleic Acids Res. 19: 6565-6572.
Henikoff and Henikoff (1992) Proc. Natl. Acad. Sci. USA 89:10915.
Hetherington et al. (1998) J. Exp. Bot. 49: 1173-1181.
Higgins et al. (1996) Methods Enzymol. 266: 383-402.
Higgins and Sharp (1988) Gene 73: 237-244.
Jaglo et al. (2001) Plant Physiol. 127: 910-917.
Kashima et al. (1985) Nature 313: 402-404.
Kim et al. (2001) Plant J. 25: 247-259.
Kyozuka and Shimamoto (2002) Plant Cell Physiol. 43: 130-135.
Larkin et al. (2007) Bioinformatics 23: 2947-2948
Lin et al. (1991) Nature 353: 569-571.
Mandel et al. (1992) Cell 71-133-143.
Manzara et al. (1993) Plant Molec. Biol. 21: 69-88.
Marcelis and Baan Hofman-Eijer (1995) Physiologia Plantarum 93 476-483.
Meyers (1995) Molecular Biology and Biotechnology, Wiley VCH, New York, N.Y., p 856-853.
Mount (2001), in Bioinformatics: Sequence and Genome Analysis, Cold Spring Harbor Laboratory Press, Cold Spring Harbor, N.Y., p. 543.
Müller et al. (2001) Plant J. 28: 169-179.
Nandi et al. (2000) Curr. Biol. 10: 215-218.
Nelson and Langdale (1989) Plant Cell 1: 3-13.
Peng et al. (1997) Genes Development 11: 3194-3205).
Peng et al. (1999) Nature: 400: 256-261.
Periago et al. (2007) J. Agric. Food Chem. 55: 8825-8829.
Piechulla et al. (1987) Plant Physiol. 84: 911-917.
Porra et al. (1989) Biochim. Biophys. Acta: 975: 384-394.
Ratcliffe et al. (2001) Plant Physiol. 126: 122-132.
Riechmann et al. (2000) Science 290: 2105-2110.
Riechmann and Ratcliffe (2000) Curr. Opin. Plant Biol. 3: 423-434.
Rieger et al. (1976) Glossary of Genetics and Cytogenetics: Classical and Molecular, 4th ed., Springer Verlag, Berlin.
Robson et al. (2001) Plant J. 28: 619-631.
Rossini et al. (2001) Plant Cell 13: 1231-1244. Russin and Trivett (2001) Vacuum-Microwave combination for processing plant tissue for electron microscopy In R T Giberson, R S Demaree, eds, Microwave: techniques and protocols. Humana Press, Totowa, N. J.
Sadowski et al. (1988) Nature 335: 563-564.
Sambrook et al. (1989) Molecular Cloning: A Laboratory Manual, 2nd Ed., Cold Spring Harbor Laboratory, Cold Spring Harbor, N.Y.
Shpaer (1997) Methods Mol. Biol. 70: 173-187.
Simpson et al. (1976) Austral. J. Plant Physiol. 3: 575-587.
Smillie et al. (1999) J. Exp. Bot. 50: 707-718.
Smith et al. (1992) Protein Engineering 5: 35-51.
Sonnhammer et al. (1997) Proteins 28: 405-420.
Southgate (1976) Determination of food carbohydrates Ed 178. Applied Science Publishers, Barking, Essex (UK).
Sugita and Gruissem (1987) Proc. Natl. Acad. Sci. (USA) 84: 7104-7108.
Suzuki et al. (2001) Plant J. 28: 409-418.
Thompson et al. (1994) Nucleic Acids Res. 22: 4673-4680.
Wanner and Gruissem (1991) Plant Cell 3: 1289-1303.
Waters et al. (2008) Plant J. 432-444.
Weigel and Nilsson (1995) Nature 377: 482-500.
Whiley A W, Schaffer B, Lara S P (1992) Tree Physiol. 11: 85-94.
Wu (ed.) Meth. Enzymol. (1993) vol. 217, Academic Press.
Xu et al. (2001) Proc. Natl. Acad. Sci. USA 98: 15089-15094.
Yasumura et al. (2005) Plant Cell 17: 1894-1907.

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

What is claimed is:

1. A transgenic plant comprising a stably integrated, recombinant polynucleotide comprising a promoter that is functional in plant cells and that is operably linked to a nucleic acid sequence that encodes a polypeptide having an amino acid percentage identity with SEQ ID NOs: 2, 4, 6, 8, 10, 12, 14, 16 or 18, or domains SEQ ID NO: 19-36, or consensus sequences SEQ ID NOs: 43 or 44; wherein
said transgenic plant is selected from a population of transgenic plants comprising said recombinant polynucleotide by screening the transgenic plants in said population and that express said polypeptide for an enhanced trait in a plant organ as compared to the plant organ of a control plant that does not have said recombinant polynucleotide;
wherein the amino acid percentage identity is selected from the group consisting of at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100%; and
wherein the enhanced trait is selected from group of enhanced traits consisting of earlier chloroplast development, darker green color when the transgenic plant develops in the absence of light, darker green color when the transgenic plant develops in low light, darker green color of a plant organ when the plant organ of the transgenic plant develops in the absence of light, darker green color of a plant organ when the plant organ of the transgenic plant develops in low light, larger chloroplasts, more extensive chloroplast thylakoid granal development, more carbohydrate levels, and more elevated chlorophyll levels, as compared to the control plant.

2. The transgenic plant of claim 1, wherein the polypeptide has an amino acid sequence with at least 81% identity to SEQ ID NO: 21 and at least 63% identity to SEQ ID NO: 22.

3. The transgenic plant of claim 1, wherein the polypeptide comprises a consensus sequence selected from the group consisting of SEQ ID NO: 43 and SEQ ID NO: 44.

4. The transgenic plant of claim 1, wherein the carbohydrate is a sugar.

5. The transgenic plant of claim 1, wherein the carbohydrate is starch.

6. The transgenic plant of claim 1, wherein the plant organ is a fruit of the transgenic plant.

7. The transgenic plant of claim 1, wherein the plant organ is a leaf, root or stem.

8. The transgenic plant of claim 1, wherein the plant organ is a transgenic seed.

9. The transgenic plant of claim 1, wherein the transgenic plant is a tomato plant.

10. The transgenic plant of claim 1, wherein the promoter is a fruit-enhanced promoter.

11. A method for producing a transgenic plant having an enhanced trait selected from the group consisting of increased carbohydrate in a plant organ, and increased chlorophyll in a plant organ, as compared to a control plant; the method steps comprising:
introducing in a target plant a recombinant polynucleotide comprising a promoter that is functional in plant cells and that is operably linked to a nucleic acid sequence that encodes a polypeptide having an amino acid percentage identity with SEQ ID NOs: 2, 4, 6, 8, 10, 12, 14, 16 or 18, or domains SEQ ID NO: 19-36, or consensus sequences SEQ ID NOs: 43 or 44, wherein:
wherein the amino acid percentage identity is selected from the group consisting of at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100%; and
said transgenic plant is selected from a population of transgenic plants comprising said recombinant DNA by screening the transgenic plants in said population and that express said polypeptide for an enhanced trait in a plant organ as compared to a control plant that does not have said recombinant DNA; and
wherein said enhanced trait is selected from group of enhanced traits consisting of earlier chloroplast development, darker green color when the transgenic plant develops in the absence of light, darker green color when the transgenic plant develops in low light, darker green color of a plant organ when the plant organ of the transgenic plant develops in the absence of light, darker green color of a plant organ when the plant organ of the transgenic plant develops in low light, larger chloroplasts, more extensive chloroplast thylakoid granal development, more carbohydrate levels, and more elevated chlorophyll levels, as compared to the control plant.

12. The method of claim 11, wherein the polypeptide has an amino acid sequence with at least 81% identity to SEQ ID NO: 21 and at least 63% identity to SEQ ID NO: 22.

13. The method of claim 11, wherein the polypeptide comprises a consensus sequence selected from the group consisting of SEQ ID NO: 43 and SEQ ID NO: 44.

14. The method of claim 11, wherein the carbohydrate is a sugar.

15. The method of claim 11, wherein the carbohydrate is starch.

16. The method of claim 11, wherein the plant organ is a fruit of the transgenic plant.

17. The method of claim 11, wherein the plant organ is a leaf, root or stem.

18. The method of claim 11, wherein the plant organ is a transgenic seed.

19. The method of claim 11, wherein the transgenic plant is a tomato plant.

20. The method of claim 11, wherein the promoter is a fruit-enhanced promoter.


SEQUENCE LISTING (44 sequences)

SEQ ID NO: 1 - DNA, 1263 bp, Arabidopsis thaliana
  AtGLK1
atgttagctctgtctccggcgacaagagatggttgcgacggagcgtcagagtttcttgatacgtcgtgtggattcacgattataaacccggaggaggaggaggagtttccggatttcgctgaccacggtgatcttcttgacatcattgacttcgacgatatattcggtgtggccggagatgtgcttcctgacttggagatcgaccctgagatcttatccggggatttctccaatcacatgaacgcttcttcaacgattactacgacgtcggataagactgatagtcaaggggagactactaagggtagttcggggaaaggtgaagaagtcgtaagcaaaagagacgatgttgcggcggagacggtgacttatgacggtgacagtgaccggaaaaggaagtattcctcttcagcttcttccaagaacaatcggatcagtaacaacgaagggaagagaaaggtgaaggtggattggacaccagagctacacaggagattcgtggaggcagtggaacagttaggagtggacaaagctgttccttctcgaattctggagcttatgggagtccattgtctcactcgtcacaacgttgctagtcacctccaaaaatataggtctcatcggaaacatttgctagctcgtgaggccgaagcggctaattggacacgcaaaaggcatatctatggagtagacaccggtgctaatcttaatggtcggactaaaaatggatggcttgcaccggcacccactctcgggtttccaccaccaccacccgtggctgttgcaccgccacctgtccaccaccatcattttaggcccctgcatgtgtggggacatcccacggttgatcagtccattatgccgcatgtgtggcccaaacacttacctccgccttctaccgccatgcctaatccgccgttttgggtctccgattctccctattggcatccaatgcataacgggacgactccgtatttaccgaccgtagctacgagatttagagcaccgccagttgccggaatcccgcatgctctgccgccgcatcacacgatgtacaaaccaaatcttggatttggtggtgctcgtcctccggtagacttacatccgtcaaaagagagcgtggatgcagccataggagatgtattgacgaggccatggctgccacttccgttgggattaaatccgccggctgttgacggtgttatgacagagcttcaccgtcacggtgtctctgaggttcctccgaccgcgtcttgtgcctga

SEQ ID NO: 2 - protein, 420 aa, Arabidopsis thaliana
  AtGLK1 polypeptide, Myb-like and GCT domains 158-206 and 370-415
MLALSPATRDGCDGASEFLDTSCGFTIINPEEEEEFPDFADHGDLLDIIDFDDIFGVAGDVLPDLEIDPEILSGDFSNHMNASSTITTTSDKTDSQGETTKGSSGKGEEVVSKRDDVAAETVTYDGDSDRKRKYSSSASSKNNRISNNEGKRKVKVDWTPELHRRFVEAVEQLGVDKAVPSRILELMGVHCLTRHNVASHLQKYRSHRKHLLAREAEAANWTRKRHIYGVDTGANLNGRTKNGWLAPAPTLGFPPPPPVAVAPPPVHHHHFRPLHVWGHPTVDQSIMPHVWPKHLPPPSTAMPNPPFWVSDSPYWHPMHNGTTPYLPTVATRFRAPPVAGIPHALPPHHTMYKPNLGFGGARPPVDLHPSKESVDAAIGDVLTRPWLPLPLGLNPPAVDGVMTELHRHGVSEVPPTASCA

SEQ ID NO: 3 - DNA, 1161 bp, Arabidopsis thaliana
  AtGLK2
atgttaactgtttctccggctccagtactcatcggaaacaactcaaaggatacttacatggcggcagatttcgcagattttacgacggaagacttgccggactttacgacggtcggggatttttccgatgatcttcttgatggaatcgattactacgacgatcttttcattggtttcgatggagacgatgttttgccggatttggagatagattcggagattcttggggaatattccggtagcggaagagatgaggaacaagaaatggagggtaacacttcgacggcatcggagacatcggagagagacgttggtgtgtgtaagcaagagggtggtggtggtggtgacggtggttttagggacaaaacggtgcgtcgaggcaaacgtaaagggaagaaaagtaaagattgtttatccgatgagaacgatattaagaaaaaacctaaggtggattggacgccggagttacaccggaaatttgtacaagcggtggagcaattaggggtagacaaggcggtgccgtctcgaatcttggaaattatgaacgttaaatctctcactcgtcacaacgttgctagccatcttcagaaatataggtcacatcggaaacatctactagcgcgtgaagcagaagctgccagctggaatctccggagacatgccacggtggcagtgcccggagtaggaggaggagggaagaagccgtggacagctcctgccttaggctatcctccacacgtggcaccaatgcatcatggtcacttcaggcctttgcacgtatggggtcatcctacgtggccaaaacacaagcctaatactccggcgtctgctcatcggacgtatccaatgccggccattgcggcggctccggcatcttggccaggtcatccaccgtactggcatcagcaaccactctatccacagggatatggtatggcatcatcgaatcattcaagcatcggtgttcccacaagacaattaggacccactaatcctcccatcgacattcatccctcgaatgagagcatagatgcagctattggggacgttatatcaaagccgtggctgccgcttcctttgggactgaaaccgccgtcggttgacggtgttatgacggagttacaacgtcaaggagtttctaatgttcctcctcttccttga

SEQ ID NO: 4 - protein, 386 aa, Arabidopsis thaliana
  AtGLK2 polypeptide, Myb-like and GCT domains 152-200 and 339-384
MLTVSPAPVLIGNNSKDTYMAADFADFTTEDLPDFTTVGDFSDDLLDGIDYYDDLFIGFDGDDVLPDLEIDSEILGEYSGSGRDEEQEMEGNTSTASETSERDVGVCKQEGGGGGDGGFRDKTVRRGKRKGKKSKDCLSDENDIKKKPKVDWTPELHRKFVQAVEQLGVDKAVPSRILEIMNVKSLTRHNVASHLQKYRSHRKHLLAREAEAASWNLRRHATVAVPGVGGGGKKPWTAPALGYPPHVAPMHHGHFRPLHVWGHPTWPKHKPNTPASAHRTYPMPAIAAAPASWPGHPPYWHQQPLYPQGYGMASSNHSSIGVPTRQLGPTNPPIDIHPSNESIDAAIGDVISKPWLPLPLGLKPPSVDGVMTELQRQGVSNVPPLP

SEQ ID NO: 5 - DNA, 1326 bp, Glycine max
  G5296
atgttggcggtgtcacctttgaggagcacaagagatgaagggcaaggagagatgatggagagtttctcgattggaaccgatgattttgctgacctttcagaagggaacttgcttgaaagcatcaacttcgatgatctcttcatgggaatcaatgacgatgaagatgtcttgccggatctggagatggaccctgagatgcttgctgagttctccctcagtactgaggaatcagatatggcctcatcatcagtttcagtggaaaataacaacaaatctgcagataacaacaacaacaatgatgggaataatatagttactactgagaaacaagatgaggttattgttatagcagccaattcttcttctgattcgggttcgagtcgaggggaggagattgtaagcaagagtgatgaatcagtggtgatgaatccatcccgtaaggaaagtgagaaaggaagaaaatcatcaaatcatgcagcaaggaataataatcctcaggggaagagaaaggttaaggtggattggaccccagaattacacaggcgattcgtgcaagcagtggagcagcttggagtggataaggctgtgccttcaaggattttggagattatgggaattgactgtctcacccgccataacattgcaagccaccttcaaaaatatagatcgcataggaagcatttgctagcgcgtgaagctgaagcagcaaggtggagtcaaaggaaacaattgttggcagcagcaggagtaggtagaggaggaggaagcaagagagaagtgaacccttggcttacaccaaccatgggtttccctcccatgacatcaatgcaccattttagacctttacatgtatgggggcatcaaaccatggaccagtccttcatgcacatgtggcctaaacatccaccatacttgccgtcaccgccggtatggccgccacaaacagctccgtctccaccggcacccgaccctctatattggcaccaacaccaacgggctccaaacgcgccaacccgaggaacaccgtgttttccacaacctctgacaaccacgagatttggctctcaaactgttcccggaatcccaccacgccatgcaatgtaccaaatactagatccaggcattggcatcccggccagccaaacgccacctcgacccctcgtcgactttcatccgtcaaaggagagcatagacgcggctattagtgatgttctatcaaaaccatggctgccactacctcttggccttaaagctccagcacttgatggtgtaatgggtgaattacaaagacaagggattcccaaaatccctccctcttgtgcttga

SEQ ID NO: 6 - protein, 441 aa, Glycine max
  G5296 polypeptide, Myb-like and GCT domains 175-223 and 393-438
MLAVSPLRSTRDEGQGEMMESFSIGTDDFADLSEGNLLESINFDDLFMGINDDEDVLPDLEMDPEMLAEFSLSTEESDMASSSVSVENNNKSADNNNNNDGNNIVTTEKQDEVIVIAANSSSDSGSSRGEEIVSKSDESVVMNPSRKESEKGRKSSNHAARNNNPQGKRKVKVDWTPELHRRFVQAVEQLGVDKAVPSRILEIMGIDCLTRHNIASHLQKYRSHRKHLLAREAEAARWSQRKQLLAAAGVGRGGGSKREVNPWLTPTMGFPPMTSMHHFRPLHVWGHQTMDQSFMHMWPKHPPYLPSPPVWPPQTAPSPPAPDPLYWHQHQRAPNAPTRGTPCFPQPLTTTRFGSQTVPGIPPRHAMYQILDPGIGIPASQTPPRPLVDFHPSKESIDAAISDVLSKPWLPLPLGLKAPALDGVMGELQRQGIPKIPPSCA

SEQ ID NO: 7 - DNA, 1368 bp, Oryza sativa
  G5290
atgcttgccgtgtcgccggcgatgtgccccgacattgaggaccgcgccgcggtggccggcgatgctggcatggaggtcgtcgggatgtcgtcggacgacatggatcagttcgacttctccgtcgatgacatagacttcggggacttcttcctgaggctggaggacggtgatgtgctcccggacctcgaggtcgacccggccgagatcttcaccgacttcgaggcaatcgcgacgagtggaggcgaaggtgtgcaggaccaggaggtgcccaccgtcgagctcttggcgcctgcggacgacgtcggtgtgctggatccgtgcggcgatgtcgtcgtcgggaaggagaacgcggcgtttgccggggctggagaggagaagggagggtgtaaccaggacgatgatgcgggggaagcgaatgccgacgatggagccgcggcggttgaggccaagtcttcgtcgccgtcatcgtcgacgtcgtcgtcgcaggaggctgagagccggcacaagtcatccagcaagagctcccatgggaagaagaaagcgaaggtggactggacgcctgagcttcaccggaggttcgtgcaggcggtggagcagctcggcatcgacaaggccgtgccgtcgaggatacttgagatcatggggatcgactcgctcacccggcacaacatagccagccatcttcagaagtaccggtcacacagaaaacacatgattgcgagagaggcggaggcagcgagttggacccaacggcggcagatttacgccgccggtggaggtgctgttgcgaagaggccggagtccaacgcgtggaccgtgccaaccattggcttccctcctcctccgccaccaccaccatcaccggctccgatgcaacattttgctcgcccgttgcatgtttggggccacccgacgatggacccgtcccgagttccagtgtggccaccgcggcacctcgttccccgtggcccggcgccaccatgggttccaccgccgccgccgtcggaccctgctttctggcaccacccttacatgagggggccagcacatgtgccaactcaagggacaccttgcatggcgatgcccatgccagctgcgagatttcctgctccaccggtgccaggagttgtcccgtgtccaatgtataggccattgactccaccagcactgacgagcaagaatcagcaggacgcacagcttcaactccaggttcaaccatcaagcgagagcatcgacgcagctatcggtgatgttttatcgaaaccgtggttgcctttgcctcttggactgaagccaccttcagtggacagtgtgatgggcgagctgcagaggcaaggcgtagcaaatgttcctccagcgtgtggatga

SEQ ID NO: 8 - protein, 455 aa, Oryza sativa
  G5290 polypeptide, Myb-like and GCT domains 185-233 and 407-452
MLAVSPAMCPDIEDRAAVAGDAGMEVVGMSSDDMDQFDFSVDDIDFGDFFLRLEDGDVLPDLEVDPAEIFTDFEAIATSGGEGVQDQEVPTVELLAPADDVGVLDPCGDVVVGKENAAFAGAGEEKGGCNQDDDAGEANADDGAAAVEAKSSSPSSSTSSSQEAESRHKSSSKSSHGKKKAKVDWTPELHRRFVQAVEQLGIDKAVPSRILEIMGIDSLTRHNIASHLQKYRSHRKHMIAREAEAASWTQRRQIYAAGGGAVAKRPESNAWTVPTIGFPPPPPPPPSPAPMQHFARPLHVWGHPTMDPSRVPVWPPRHLVPRGPAPPWVPPPPPSDPAFWHHPYMRGPAHVPTQGTPCMAMPMPAARFPAPPVPGVVPCPMYRPLTPPALTSKNQQDAQLQLQVQPSSESIDAAIGDVLSKPWLPLPLGLKPPSVDSVMGELQRQGVANVPPACG

SEQ ID NO: 9 - DNA, 1620 bp, Oryza sativa
  G5291
atgcttgaggtgtccacgctgcgaagccctaaggcggatcagcgggcgggcgtcggcggccaccatgtcgtcggcttcgtcccggcgccgccgtcgccggccgacgtcgccgacgaggtcgacgcgttcatcgtcgacgacagctgcctgctcgagtacatcgacttcagctgctgcgacgtgccgttcttccacgccgacgacggcgacatcctcccggacctcgaggtcgaccccacggagctcctcgccgagttcgccagctccccggacgacgagccgccgccgacgacgtcggctccgggccccggcgagccagctgctgctgcaggagccaaggaagacgtgaaggaagatggagccgccgccgccgccgccgccgccgccgctgactacgacgggtcgccgccgccaccgcgggggaagaagaagaaggacgacgaggaaaggtcgtcgtcgttgccggaggagaaagacgcgaagaacggcggcggcgacgaggtcctgagcgcggtgacgacggaggattcctcggccggtgccgccaagtcgtgctcgccgtcggcagagggccacagcaagaggaagccgtcgtcgtcgtcgtcatcggcggcggccggcaagaactctcacggcaagcgcaaggtgaaggtggactggacgccggagttgcaccggcggttcgtgcaggcggtggagcagctcgggatagacaaggccgtgccgtccaggatcctggagctcatgggcatcgagtgcctcactcgccacaacatcgccagccatctccagaaatatcggtcgcacaggaaacatctgatggcgagggaggcggaggcggcgagctggacgcagaagcggcagatgtacaccgccgccgccgccgccgccgcggtggcagccggcggcgggccaaggaaggacgccgccgccgccactgcggcggtggccccgtgggtcatgccgaccatcggtttccctccgccgcacgcggcggcgatggtgcctcccccgccgcaccctccaccgttctgccggccgccgctgcacgtgtggggccacccgaccgccggcgtcgagccgaccaccgcggcggcgccaccaccaccctcgccgcacgcgcagccgccgttgctgcccgtctggccgcgccacctggcgccgccgccgccgccgctgccggcggcgtgggcgcacggccaccagccggcgccggtggacccggcggcgtactggcagcaacagtataacgcggcgaggaagtggggcccgcaggcagtgacaccggggacgccgtgtatgccgccaccgttgcctccagccgccatgttgcagaggtttcctgtaccgccggtgcctggaatggtgccgcaccccatgtacagaccgataccgccgccgtcaccgccgcaggggaataaactcgctgccttgcagcttcagcttgatgcccacccgtctaaggagagcatagacgcagccatcggagatgttttagtgaagccatggctgccgcttcccctcggcctcaagccaccgtcgctggacagcgtcatgtctgagctgcacaagcagggcatccccaaggtgccaccggcggcgagcggtgccgccggctga

SEQ ID NO: 10 - protein, 539 aa, Oryza sativa
  G5291 polypeptide, Myb-like and GCT domains 220-268 and 487-532
MLEVSTLRSPKADQRAGVGGHHVVGFVPAPPSPADVADEVDAFIVDDSCLLEYIDFSCCDVPFFHADDGDILPDLEVDPTELLAEFASSPDDEPPPTTSAPGPGEPAAAAGAKEDVKEDGAAAAAAAAAADYDGSPPPPRGKKKKDDEERSSSLPEEKDAKNGGGDEVLSAVTTEDSSAGAAKSCSPSAEGHSKRKPSSSSSSAAAGKNSHGKRKVKVDWTPELHRRFVQAVEQLGIDKAVPSRILELMGIECLTRHNIASHLQKYRSHRKHLMAREAEAASWTQKRQMYTAAAAAAAVAAGGGPRKDAAAATAAVAPWVMPTIGFPPPHAAAMVPPPPHPPPFCRPPLHVWGHPTAGVEPTTAAAPPPPSPHAQPPLLPVWPRHLAPPPPPLPAAWAHGHQPAPVDPAAYWQQQYNAARKWGPQAVTPGTPCMPPPLPPAAMLQRFPVPPVPGMVPHPMYRPIPPPSPPQGNKLAALQLQLDAHPSKESIDAAIGDVLVKPWLPLPLGLKPPSLDSVMSELHKQGIPKVPPAASGAAG

SEQ ID NO: 11 - DNA, 1554 bp, Physcomitrella patens
  G5294
atggcgatggacatggcgaggatagatgaatcaaccgccgtcgaggtcaactcgctttcgcttgtgcattgcgtgttggatgggttgcccgattcgccttgcttgaaatccagcccgacgtcattcgaggaggctgtggcggaagggaggtcggtgttcggggacgaggaggacatcatcaacaacagcaacgaccaggacaactcgtcctcgtgcggtgcagtggtcaccacccacgaagatttcgccgagtgcttgaattttgtgacggaggcggagtgtggagatgtgggtgtgcggtgtttcgaggattttgacaagctgccggactgcggcgacgagggggagactagcaaagcggaggaggaggggtgtgtacgaataggcggaggaggggagcagggcgagctgttagagtctgtgagccttgactgtagtaggaattcggagaatttagagcttcgggatctcggggaattgtgggaagggtcggaacggcctgactcagtgccagggaacgaggtgggtgaggaggaggcgttgctgttggcggaggcggccaaggcgacgggcgatgtcgtgtcggcctcggatagtggagaatgtagcagtgtcgataggaaggacaatcaagccagtccgaaatccagtaaaaatgcagcgccggggaagaagaaggccaaggtggactggacgcctgagcttcaccggcgcttcgtccatgcagtggagcagctgggtgtggagaaagcctatccatcgcgtatcctagaactgatgggcgtgcaatgcttgactcggcacaacatcgccagtcacttgcagaagtaccggtcccaccgtcgccacctcgcagctcgagaagcagaggccgcgtcctggacgcatcgtcgcacatacactcaggctccctggcctcgtagctcacggcgcgatggcctcccttatcttgtacctatacacacccctcacatacaacctcggccttccatggccatggcaatgcaaccgcagcttcagacgccgcatcatccgatatccactcctctcaaggtctggggctaccccacagtagatcattcaaatgtacatatgtggcagcaacctgctgtggcgaccccatcttactggcaagctgccgatggctcatactggcaacatcccgcgaccggttacgacgctttctcagctcgtgcctgctactcgcatcccatgcagcgagttcctgtaacgaccacgcatgcgggtttaccaattgtggcgccaggatttcctgacgagagctgctactacggcgacgacatgcttgcaggctccatgtatctatgtaaccaatcatatgatagtgaaataggacgagctgcgggtgttgctgcgtgcagcaagccgatagagacgcatttgtccaaagaggtgttggatgcggccattggcgaagctctcgccaatccctggactcccccacctctgggtctgaagccaccatccatggagggcgtcattgcagagcttcagcggcaggggatcaacactgtgcctccttcaacttgttag

SEQ ID NO: 12 - protein, 517 aa, Physcomitrella patens
  G5294 polypeptide, Myb-like and GCT domains 231-279 and 469-514
MAMDMARIDESTAVEVNSLSLVHCVLDGLPDSPCLKSSPTSFEEAVAEGRSVFGDEEDIINNSNDQDNSSSCGAVVTTHEDFAECLNFVTEAECGDVGVRCFEDFDKLPDCGDEGETSKAEEEGCVRIGGGGEQGELLESVSLDCSRNSENLELRDLGELWEGSERPDSVPGNEVGEEEALLLAEAAKATGDVVSASDSGECSSVDRKDNQASPKSSKNAAPGKKKAKVDWTPELHRRFVHAVEQLGVEKAYPSRILELMGVQCLTRHNIASHLQKYRSHRRHLAAREAEAASWTHRRTYTQAPWPRSSRRDGLPYLVPIHTPHIQPRPSMAMAMQPQLQTPHHPISTPLKVWGYPTVDHSNVHMWQQPAVATPSYWQAADGSYWQHPATGYDAFSARACYSHPMQRVPVTTTHAGLPIVAPGFPDESCYYGDDMLAGSMYLCNQSYDSEIGRAAGVAACSKPIETHLSKEVLDAAIGEALANPWTPPPLGLKPPSMEGVIAELQRQGINTVPPSTC

SEQ ID NO: 13 - DNA, 1536 bp, Physcomitrella patens
  G5295
atggcgatggacatggcgaggatagaagtcgagcctcttgtcgaagtccacaacaacaccaacaacttgcttgtgcattgcgtgctcgatgctttgccggattcttcaccttgcttgaaatccagtgagacttcgtttgaagctgtggttgtaaaggaggacgaggagggagggaggccgctgtttggcaagcctgagctccaccctgcctcaccctcgagtgatacagcggctgctgcaaatggtgaattcgctagatgcttgaattttgtgacggaggctgattgtggagatgtaggcgtacaatgttttgaggattttggtacgttgccggactgtggtgaggcggggataagcagggaagagggtggaggtagagacggggagcaggtggagttgttagagtctatgagcctcgatggtagtaggaattcggagaatttagagctaggggagctcggcgaattgttgcaggggtcggagacgctggattcggttcctgggaacgaggttggggaggaggaggcgctgctgttggcgaaagcggcaaaggcgacgggcgttgttgtttcggcctccgatagtggtgaatgtagcagtgtcgataggaaggaaaatcaacaaagtccgaaatcatgtaaaagtgccgcaccggggaagaagaaggcgaaggtcgattggacgccggagcttcatcggcgctttgtccacgcggtggaacagcttggagtggagaaagcttttccctcgcgcatactagaactgatgggagtacaatgtctcacccggcacaatatcgccagtcatttgcagaagtatcgctcgcatcgtcgccatcttgcagccagggaagccgaggcagcatcctggactcatcgtcgagcgtacacccagatgccctggtctcgaagttcacggcgcgatggccttccttatcttgtacccttacacacccctcacatacaacctcgcccatccatggtcatggcaatgcaaccacagcttcagacgcagcacaccccggtgtcgacgcctcttaaggtgtgggggtatcctacagtagatcattcaagtgtacacatgtggcagcaacctgcagtggcgaccccatcgtattggcaagcccccgatggctcttactggcagcatcctgccaccaattatgatgcgtattcagctcgcgcttgttatccccatcccatgcgagtttcgttaggcactacgcatgctggctctccaatgatggctccaggatttcctgacgagagctactacggtgaagatgttcttgcagctaccatgtatctttgtaaccaatcatatgacagtgaattaggacgagctgcgggcgtcgctgcgtgcagtaaaccaccggagacgcatttatcgaaggaggttcttgatgcagccatcggagaagcgcttgctaacccttggactcccccgcccctgggactgaagccgccgtctatggagggagtaatcgcagagcttcagcggcagggaatcaacactgtgcctccctctacctgttag

SEQ ID NO: 14 - protein, 511 aa, Physcomitrella patens
  G5295 polypeptide, Myb-like and GCT domains 227-275 and 463-508
MAMDMARIEVEPLVEVHNNTNNLLVHCVLDALPDSSPCLKSSETSFEAVVVKEDEEGGRPLFGKPELHPASPSSDTAAAANGEFARCLNFVTEADCGDVGVQCFEDFGTLPDCGEAGISREEGGGRDGEQVELLESMSLDGSRNSENLELGELGELLQGSETLDSVPGNEVGEEEALLLAKAAKATGVVVSASDSGECSSVDRKENQQSPKSCKSAAPGKKKAKVDWTPELHRRFVHAVEQLGVEKAFPSRILELMGVQCLTRHNIASHLQKYRSHRRHLAAREAEAASWTHRRAYTQMPWSRSSRRDGLPYLVPLHTPHIQPRPSMVMAMQPQLQTQHTPVSTPLKVWGYPTVDHSSVHMWQQPAVATPSYWQAPDGSYWQHPATNYDAYSARACYPHPMRVSLGTTHAGSPMMAPGFPDESYYGEDVLAATMYLCNQSYDSELGRAAGVAACSKPPETHLSKEVLDAAIGEALANPWTPPPLGLKPPSMEGVIAELQRQGINTVPPSTC

SEQ ID NO: 15 - DNA, 1386 bp, Zea mays
  G5292
atgcttgaggtgtcgacgctgcgcggccctactagcagcggcagcaaggcggagcagcactgcggcggcggcggcggcttcgtcggcgaccaccatgtggtgttcccgacgtccggcgactgcttcgccatggtggacgacaacctcctggactacatcgacttcagctgcgacgtgcccttcttcgacgctgacggggacatcctccccgacctggaggtagacaccacggagctcctcgccgagttctcgtccacccctcctgcggacgacctgctggcagtggcagtattcggcgccgacgaccagccggcggcggcagtagcacaagagaagccgtcgtcgtcgttggagcaaacatgtggtgacgacaaaggtgtagcagtagccgccgccagaagaaagctgcagacgacgacgacgacgacgacgacggaggaggaggattcttctcctgccgggtccggggccaacaagtcgtcggcgtcggcagagggccacagcagcaagaagaagtcggcgggcaagaactccaacggcggcaagcgcaaggtgaaggtggactggacgccggagctgcaccggcggttcgtgcaggcggtggagcagctgggcatcgacaaggccgtgccgtccaggatcctggagatcatgggcacggactgcctcacaaggcacaacattgccagccacctccagaagtaccggtcgcacagaaagcacctgatggcgcgggaggcggaggccgccacctgggcgcagaagcgccacatgtacgcgccgccagctccaaggacgacgacgacgacggacgccgccaggccgccgtgggtggtgccgacgaccatcgggttcccgccgccgcgcttctgccgcccgctgcacgtgtggggccacccgccgccgcacgccgccgcggctgaagcagcagcggcgactcccatgctgcccgtgtggccgcgtcacctggcgccgccccggcacctggcgccgtgggcgcacccgacgccggtggacccggcgttctggcaccagcagtacagcgctgccaggaaatggggcccacaggcagccgccgtgacgcaagggacgccatgcgtgccgctgccgaggtttccggtgcctcaccccatctacagcagaccggcgatggtacctccgccgccaagcaccaccaagctagctcaactgcatctggagctccaagcgcacccgtccaaggagagcatcgacgcagccatcggagatgttttagtgaagccatggctgccgcttccactggggctcaagccgccgtcgctcgacagcgtcatgtcggagctgcacaagcaaggcgtaccaaaaatcccaccggcggctgccaccaccaccggcgccaccggatga

SEQ ID NO: 16 - protein, 461 aa, Zea mays
  G5292 polypeptide, Myb-like and GCT domains 189-237 and 406-451
MLEVSTLRGPTSSGSKAEQHCGGGGGFVGDHHVVFPTSGDCFAMVDDNLLDYIDFSCDVPFFDADGDILPDLEVDTTELLAEFSSTPPADDLLAVAVFGADDQPAAAVAQEKPSSSLEQTCGDDKGVAVAAARRKLQTTTTTTTTEEEDSSPAGSGANKSSASAEGHSSKKKSAGKNSNGGKRKVKVDWTPELHRRFVQAVEQLGIDKAVPSRILEIMGTDCLTRHNIASHLQKYRSHRKHLMAREAEAATWAQKRHMYAPPAPRTTTTTDAARPPWVVPTTIGFPPPRFCRPLHVWGHPPPHAAAAEAAAATPMLPVWPRHLAPPRHLAPWAHPTPVDPAFWHQQYSAARKWGPQAAAVTQGTPCVPLPRFPVPHPIYSRPAMVPPPPSTTKLAQLHLELQAHPSKESIDAAIGDVLVKPWLPLPLGLKPPSLDSVMSELHKQGVPKIPPAAATTTGATG

SEQ ID NO: 17 - DNA, 1428 bp, Zea mays
  G5293
atgcttgcagtgtcgccgtcgccggtgcggtgtgccgatgcggaggagtgcggcggaggaggcgccagcaaggaaatggaggagaccgccgtcgggcctgtgtccgactcggacctggatttcgacttcacggtcgacgacatagacttcggggacttcttcctcaggctagacgacggggatgacgcgctgccgggcctcgaggtcgaccctgccgagatcgtcttcgctgacttcgaggcaatcgccaccgccggcggcgatggcggcgtcacggaccaggaggtgcccagtgtcctgccctttgcggacgcggcgcacataggcgccgtggatccgtgttgtggtgtccttggcgaggacaacgacgcagcgtgcgcagacgtggaagaagggaaaggggagtgcgaccatgccgacgaggtagcagccgccggtaataataatagcgactccggtgaggccggctgtggaggagcctttgccggcgaaaaatcaccgtcgtcgacggcatcgtcgtcgcaggaggctgagagccggcgcaaggtgtccaagaagcactcccaagggaagaagaaagcaaaggtggattggacgccggagcttcaccggagattcgttcaggcggtggaggagctgggcatcgacaaggcggtgccgtccaggatcctcgagatcatggggatcgactccctcacgcggcataacatagccagccatctgcagaagtaccgttcccacaggaagcacatgcttgcgagggaggtggaggcagcgacgtggacgacgcaccggcggccgatgtacgctgcccccagcggcgccgtgaagaggcccgactctaacgcgtggaccgtgccgaccatcggtttccctccgccggcggggacccctcctcgtccggtgcagcacttcgggaggccactgcacgtctggggccatccgagtccgacgccagcggtggagtcaccccgggtgccaatgtggcctcggcatctcgccccccgcgccccgccgccgccgccgtgggctccgccaccgccagctgacccggcgtcgttctggcaccatgcttacatgagggggcctgctgcccatatgccagaccaggtggcggtgactccatgcgtggcagtgccaatggcagcagcgcgtttccctgctccacacgtgaggggttctttgccatggccacctccgatgtacagacctctcgttcctccagcactcgcaggcaagagccagcaagacgcgctgtttcagctacagatacagccatcaagcgagagcatagatgcagcaataggtgatgtcttaacgaagccgtggctgccgctgcccctcggactgaagcccccttcggtagacagtgtcatgggcgagctgcagaggcaaggcgtagcgaatgtgccgcaagcttgtggatga

SEQ ID NO: 18 - protein, 475 aa, Zea mays
  G5293 polypeptide, Myb-like and GCT domains 198-246 and 427-472
MLAVSPSPVRCADAEECGGGGASKEMEETAVGPVSDSDLDFDFTVDDIDFGDFFLRLDDGDDALPGLEVDPAEIVFADFEAIATAGGDGGVTDQEVPSVLPFADAAHIGAVDPCCGVLGEDNDAACADVEEGKGECDHADEVAAAGNNNSDSGEAGCGGAFAGEKSPSSTASSSQEAESRRKVSKKHSQGKKKAKVDWTPELHRRFVQAVEELGIDKAVPSRILEIMGIDSLTRHNIASHLQKYRSHRKHMLAREVEAATWTTHRRPMYAAPSGAVKRPDSNAWTVPTIGFPPPAGTPPRPVQHFGRPLHVWGHPSPTPAVESPRVPMWPRHLAPRAPPPPPWAPPPPADPASFWHHAYMRGPAAHMPDQVAVTPCVAVPMAAARFPAPHVRGSLPWPPPMYRPLVPPALAGKSQQDALFQLQIQPSSESIDAAIGDVLTKPWLPLPLGLKPPSVDSVMGELQRQGVANVPQACG

SEQ ID NO: 19 - protein, 49 aa, Arabidopsis thaliana
  AtGLK1 Myb-like DNA binding domain
WTPELHRRFVEAVEQLGVDKAVPSRILELMGVHCLTRHNVASHLQKYRS

SEQ ID NO: 20 - protein, 46 aa, Arabidopsis thaliana
  AtGLK1 GCT domain
SKESVDAAIGDVLTRPWLPLPLGLNPPAVDGVMTELHRHGVSEVPP

SEQ ID NO: 21 - protein, 49 aa, Arabidopsis thaliana
  AtGLK2 Myb-like DNA binding domain
WTPELHRKFVQAVEQLGVDKAVPSRILEIMNVKSLTRHNVASHLQKYRS

SEQ ID NO: 22 - protein, 46 aa, Arabidopsis thaliana
  AtGLK2 GCT domain
SNESIDAAIGDVISKPWLPLPLGLKPPSVDGVMTELQRQGVSNVPP

SEQ ID NO: 23 - protein, 49 aa, Arabidopsis thaliana
  G5296 Myb-like DNA binding domain
WTPELHRRFVQAVEQLGVDKAVPSRILEIMGIDCLTRHNIASHLQKYRS

SEQ ID NO: 24 - protein, 46 aa, Arabidopsis thaliana
  G5296 GCT domain
SKESIDAAISDVLSKPWLPLPLGLKAPALDGVMGELQRQGIPKIPP

SEQ ID NO: 25 - protein, 49 aa, Oryza sativa
  G5290 Myb-like DNA binding domain
WTPELHRRFVQAVEQLGIDKAVPSRILEIMGIDSLTRHNIASHLQKYRS

SEQ ID NO: 26 - protein, 46 aa, Oryza sativa
  G5290 GCT domain
SSESIDAAIGDVLSKPWLPLPLGLKPPSVDSVMGELQRQGVANVPP

SEQ ID NO: 27 - protein, 49 aa, Oryza sativa
  G5291 Myb-like DNA binding domain
WTPELHRRFVQAVEQLGIDKAVPSRILELMGIECLTRHNIASHLQKYRS

SEQ ID NO: 28 - protein, 46 aa, Oryza sativa
  G5291 GCT domain
SKESIDAAIGDVLVKPWLPLPLGLKPPSLDSVMSELHKQGIPKVPP

SEQ ID NO: 29 - protein, 49 aa, Physcomitrella patens
  G5294 Myb-like DNA binding domain
WTPELHRRFVHAVEQLGVEKAYPSRILELMGVQCLTRHNIASHLQKYRS

SEQ ID NO: 30 - protein, 46 aa, Physcomitrella patens
  G5294 GCT domain
SKEVLDAAIGEALANPWTPPPLGLKPPSMEGVIAELQRQGINTVPP

SEQ ID NO: 31 - protein, 49 aa, Physcomitrella patens
  G5295 Myb-like DNA binding domain
WTPELHRRFVHAVEQLGVEKAFPSRILELMGVQCLTRHNIASHLQKYRS

SEQ ID NO: 32 - protein, 46 aa, Physcomitrella patens; identical to SEQ ID NO: 30
  G5295 GCT domain

SEQ ID NO: 33 - protein, 49 aa, Zea mays
  G5292 Myb-like DNA binding domain
WTPELHRRFVQAVEQLGIDKAVPSRILEIMGTDCLTRHNIASHLQKYRS

SEQ ID NO: 34 - protein, 46 aa, Zea mays
  G5292 GCT domain
SKESIDAAIGDVLVKPWLPLPLGLKPPSLDSVMSELHKQGVPKIPP

SEQ ID NO: 35 - protein, 49 aa, Zea mays
  G5293 Myb-like DNA binding domain
WTPELHRRFVQAVEELGIDKAVPSRILEIMGIDSLTRHNIASHLQKYRS

SEQ ID NO: 36 - protein, 46 aa, Zea mays
  G5293 GCT domain
SSESIDAAIGDVLTKPWLPLPLGLKPPSVDSVMGELQRQGVANVPQ

SEQ ID NO: 37 - DNA, 1376 bp, Arabidopsis thaliana
  P7446 opLexA::AtGLK1 nucleic acid construct
ctctatctacgattatatttggatctacaagtgaagattgatcgatgttagctctgtctccggcgacaagagatggttgcgacggagcgtcagagtttcttgatacgtcgtgtggattcacgattataaacccggaggaggaggaggagtttccggatttcgctgaccacggtgatcttcttgacatcattgacttcgacgatatattcggtgtggccggagatgtgcttcctgacttggagatcgaccctgagatcttatccggggatttctccaatcacatgaacgcttcttcaacgattactacgacgtcggataagactgatagtcaaggggagactactaagggtagttcggggaaaggtgaagaagtcgtaagcaaaagagacgatgttgcggcggagacggtgacttatgacggtgacagtgaccggaaaaggaagtattcctcttcagcttcttccaagaacaatcggatcagtaacaacgaagggaagagaaaggtgaaggtggattggacaccagagctacacaggagattcgtggaggcagtggaacagttaggagtggacaaagctgttccttctcgaattctggagcttatgggagtccattgtctcactcgtcacaacgttgctagtcacctccaaaaatataggtctcatcggaaacatttgctagctcgtgaggccgaagcggctaattggacacgcaaaaggcatatctatggagtagacaccggtgctaatcttaatggtcggactaaaaatggatggcttgcaccggcacccactctcgggtttccaccaccaccacccgtggctgttgcaccgccacctgtccaccaccatcattttaggcccctgcatgtgtggggacatcccacggttgatcagtccattatgccgcatgtgtggcccaaacacttacctccgccttctaccgccatgcctaatccgccgttttgggtctccgattctccctattggcatccaatgcataacgggacgactccgtatttaccgaccgtagctacgagatttagagcaccgccagttgccggaatcccgcatgctctgccgccgcatcacacgatgtacaaaccaaatcttggatttggtggtgctcgtcctccggtagacttacatccgtcaaaagagagcgtggatgcagccataggagatgtattgacgaggccatggctgccacttccgttgggattaaatccgccggctgttgacggtgttatgacagagcttcaccgtcacggtgtctctgaggttcctccgaccgcgtcttgtgcctgaaacgcacaagatccgtaggcaagcgagaaccaaacaaaaattcgacgacatgtctttcaattattgtac

SEQ ID NO: 38 - DNA, 1384 bp, Arabidopsis thaliana
  P5537 opLexA::AtGLK2 nucleic acid construct
atttttcaaaaaacctttagaattttcatttttttacgattccgatgttaactgtttctccggctccagtactcatcggaaacaactcaaaggatacttacatggcggcagatttcgcagattttacgacggaagacttgccggactttacgacggtcggggatttttccgatgatcttcttgatggaatcgattactacgacgatcttttcattggtttcgatggagacgatgttttgccggatttggagatagattcggagattcttggggaatattccggtagcggaagagatgaggaacaagaaatggagggtaacacttcgacggcatcggagacatcggagagagacgttggtgtgtgtaagcaagagggtggtggtggtggtgacggtggttttagggacaaaacggtgcgtcgaggcaaacgtaaagggaagaaaagtaaagattgtttatccgatgagaacgatattaagaaaaaacctaaggtggattggacgccggagttacaccggaaatttgtacaagcggtggagcaattaggggtagacaaggcggtgccgtctcgaatcttggaaattatgaacgttaaatctctcactcgtcacaacgttgctagccatcttcagaaatataggtcacatcggaaacatctactagcgcgtgaagcagaagctgccagctggaatctccggagacatgccacggtggcagtgcccggagtaggaggaggagggaagaagccgtggacagctcctgccttaggctatcctccacacgtggcaccaatgcatcatggtcacttcaggcctttgcacgtatggggtcatcctacgtggccaaaacacaagcctaatactccggcgtctgctcatcggacgtatccaatgccggccattgcggcggctccggcatcttggccaggtcatccaccgtactggcatcagcaaccactctatccacagggatatggtatggcatcatcgaatcattcaagcatcggtgttcccacaagacaattaggacccactaatcctcccatcgacattcatccctcgaatgagagcatagatgcagctattggggacgttatatcaaagccgtggctgccgcttcctttgggactgaaaccgccgtcggttgacggtgttatgacggagttacaacgtcaaggagtttctaatgttcctcctcttccttgagaaagatctccaaaatttgtcgaaatctcaaacttttaacttcatttttttggtatcttctatgtatttttgcaagggaaagacgataaatccttgttgcttgatcatatgtatttctctatatgagtgcatgtatcgaagttaagcaactttaatatatcgttataatcttcgataaa

SEQ ID NO: 39 - DNA, 255 bp, Arabidopsis thaliana
  P5287 prLTP1::m35S::oEnh::LexAGal4(GFP) driver construct
gatatgaccaaaatgattaacttgcattacagttgggaagtatcaagtaaacaacattttgtttttgtttgatatcgggaatctcaaaaccaaagtccacactagtttttggactatataatgataaaagtcagatatctactaatactagttgatcagtatattcgaaaacatgactttccaaatgtaagttatttactttttttttgctattataattaagatcaataaaaatgtctaagttttaaatcttta

SEQ ID NO: 40 - DNA, 255 bp, Solanum lycopersicum
  P5284 prRBCS3::m35S::oEnh::LexAGal4(GFP) driver construct
aaatggagtaatatggataatcaacgcaactatatagagaaaaaataatagcgctaccatatacgaaaaatagtaaaaaattataataatgattcagaataaattattaataactaaaaagcgtaaagaaataaattagagaataagtgatacaaaattggatgttaatggatacttcttataattgcttaaaaggaatacaagatgggaaataatgtgttattattattgatgtataaagaatttgtacaattt

SEQ ID NO: 41 - DNA, 255 bp, Solanum lycopersicum
  P5303 prPD::m35S::oEnh::LexAGal4(GFP) driver construct
acgtgtaatagctaccatacaagagaagtaactcgcactgtccatgtcttatgtggctcgactcagaaagcattcagggggattgataaccaccctccaaaccaactgaaccattgtgaataaccacccttcaaatcaaccgagtcctcgtgaaggacaaatatgtggttttatatacattaaattttgtttttacatgcttcctcttacttctttagttttcttgaccatatcttgcgtttttcccttctgtaa

SEQ ID NO: 42 - DNA, 802 bp, Cauliflower mosaic virus
  P6506 35S::m35S::oEnh::LexAGal4(GFP) driver construct
gcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttc

SEQ ID NO: 43 - protein, 49 aa, Arabidopsis thaliana
  misc_feature (8)..(8): Xaa can be any naturally occurring amino acid
WTPELHRXFVXAVEXLGXXKAXPSRILEXMXXXXLTRHNXASHLQKYRS

SEQ ID NO: 44 - protein, 45 aa, Arabidopsis thaliana
  misc_feature (2)..(2): Xaa can be any naturally occurring amino acid
SXEXXDAAIXXXXXXPWXPXPLGLXXPXXXXVXXELXXXGXXXXP